SEQUENCE LISTING (46 sequences)

SEQ ID NO: 1 - protein, 20 aa, Homo sapiens
YEYYHALDSDHLTKTHHDMF

SEQ ID NO: 2 - protein, 15 aa, Homo sapiens
YSFYVLDNQNLQQLW

SEQ ID NO: 3 - protein, 15 aa, Homo sapiens
TQYAVYVKAVTLTMV

SEQ ID NO: 4 - protein, 15 aa, Homo sapiens
KSESTFKNTEISFIL

SEQ ID NO: 5 - protein, 15 aa, Homo sapiens
MAPALRHLYKELMGP

SEQ ID NO: 6 - protein, 15 aa, Homo sapiens
IINDWVKTHTKGMIS

SEQ ID NO: 7 - protein, 15 aa, Homo sapiens
CRSFFAFNAGHIAGS

SEQ ID NO: 8 - protein, 15 aa, Homo sapiens
QFNLRNLYRLSKALF

SEQ ID NO: 9 - protein, 15 aa, Homo sapiens
CQYLVMRAKRKLGRH

SEQ ID NO: 10 - protein, 15 aa, Homo sapiens
IIRSFLLKVLPADSH

SEQ ID NO: 11 - protein, 20 aa, Homo sapiens
GGYVWVETQATVIYNTKNSQ

SEQ ID NO: 12 - protein, 15 aa, Homo sapiens
NMTRLPRLLVLPKFS

SEQ ID NO: 13 - protein, 15 aa, Homo sapiens
LSALTNILSAQLISH

SEQ ID NO: 14 - protein, 15 aa, Homo sapiens
LVLVNALYFNGQWKT

SEQ ID NO: 15 - protein, 15 aa, Homo sapiens
LSALTNILSAQLISH

SEQ ID NO: 16 - protein, 35 aa, Homo sapiens
FEGLKAFRGVDNKIRLFQPNLNMDRMYRSAVRATL

SEQ ID NO: 17 - protein, 36 aa, Homo sapiens
CIQQLVKLDQEWVPYSTSASLYIRPTFIGTEPSLGV

SEQ ID NO: 18 - protein, 35 aa, Homo sapiens
FVLLSPVGPYFSSGTFNPVSLWANPKYVRAWKGGT

SEQ ID NO: 19 - protein, 23 aa, Homo sapiens
DHQITEVGTMNLFLYWINEDGEE

SEQ ID NO: 20 - protein, 26 aa, Homo sapiens
MFGSGTACVVCPVSDILYKGETIHIP

SEQ ID NO: 21 - protein, 21 aa, Homo sapiens
FGKMAGSGVRQATSTASTFVK

SEQ ID NO: 22 - protein, 27 aa, Homo sapiens
RAWYREVPNTVHQFQLDITVKMGRDKV

SEQ ID NO: 23 - protein, 29 aa, Homo sapiens
ELEETIKVWKQRTHVMRFFHETEAPRPKD

SEQ ID NO: 24 - protein, 29 aa, Homo sapiens
LIEYHKNGHLSFKYVKTFNMDEYVGLPRN

SEQ ID NO: 25 - protein, 18 aa, Homo sapiens
FFKHIDIDPNNAHILDGN

SEQ ID NO: 26 - protein, 28 aa, Homo sapiens
REVMILITGAHKAFALYKAIEEGVNHMW

SEQ ID NO: 27 - protein, 22 aa, Homo sapiens
RVKTVKYFKGLMHVHNKLVDPL

SEQ ID NO: 28 - protein, 19 aa, Homo sapiens
VRKVKVVVNFAPTIQEIKS

SEQ ID NO: 29 - protein, 30 aa, Homo sapiens
WYKGEKKLFNGQQGIIIQNFSTRSILTVTN

SEQ ID NO: 30 - protein, 33 aa, Homo sapiens
GITGSADVLFSCWYLVLTLSSFTSIFYLKNAIL

SEQ ID NO: 31 - protein, 29 aa, Homo sapiens
YIIAFAKEKNLLKYIQFKTFVSSVNKHPD

SEQ ID NO: 32 - protein, 36 aa, Homo sapiens
VFDAVMVCSGHHVYPNLPKESFPGLNHFKGKCFHSR

SEQ ID NO: 33 - protein, 21 aa, Homo sapiens
VKPNVKEFTETSAIFEDGTIF

SEQ ID NO: 34 - protein, 30 aa, Homo sapiens
LRDAIKRRGDFEMDVVAMVNDTVATMISCY

SEQ ID NO: 35 - protein, 25 aa, Homo sapiens
GKYMGELVRLVLLRLVDENLLFHGE

SEQ ID NO: 36 - protein, 19 aa, Homo sapiens
DRKQIYNILSTLGLRPSTT

SEQ ID NO: 37 - protein, 21 aa, Homo sapiens
YKLHPSFKERFASVRRLTPSC

SEQ ID NO: 38 - protein, 38 aa, Homo sapiens
LIVLGYNGSLPNGDRGRRKSRFALFKRPKANGVKPSTV

SEQ ID NO: 39 - protein, 28 aa, Homo sapiens
HSISYTLSRAQTVVVEYTHDSNTDMFQI

SEQ ID NO: 40 - protein, 29 aa, Homo sapiens
ARIYAAGFDSSKNIFLGEKAAKWKTSDGQ

SEQ ID NO: 41 - protein, 25 aa, Homo sapiens
SHTPTVKHLEALRQEINAARPQCPV

SEQ ID NO: 42 - protein, 56 aa, Homo sapiens
SNGSICLDILASQWSPALTISKVLLSICSLLCDPNPDDPLVPEIARIYKTDRDKYN

SEQ ID NO: 43 - protein, 24 aa, Homo sapiens
IKDEFQFLQAQYHSLKVEYDKLAN

SEQ ID NO: 44 - protein, 23 aa, Homo sapiens
AKRLNTILAQIMPFLSQEHQQVA

SEQ ID NO: 45 - protein, 22 aa, Homo sapiens
RTPISITSSYAAPFAMMSHHEM

SEQ ID NO: 46 - protein, 28 aa, Homo sapiens
PTPRIKAELTSSAPACYALAISPDAKVC

(19) United States
(12) Patent Application Publication
(10) Pub. No.: US 2026/0345757 A1
(43) Pub. Date: Nov. 5, 2026

(54) TH2 VACCINE-BASED PREVENTION AND TREATMENT OF INFLAMMATION IN OBESITY

(71) Applicant: UNIVERSITY OF WASHINGTON, SEATTLE, WA (US)

(72) Inventors: Mary L. Disis, SEATTLE, WA (US); Denise Cecil, SEATTLE, WA (US); Lauren Corulli, SEATTLE, WA (US)

(73) Assignee: UNIVERSITY OF WASHINGTON, SEATTLE, WA (US)

(21) Appl. No.: 19/104,960

(22) PCT Filed: Aug. 14, 2023
(86) PCT No.: PCT/US23/72164

§ 371 Date: Feb. 19, 2025

Related U.S. Application Data
(60) Provisional application No. 63/371,999, filed on Aug. 19, 2022.

(51) Int. Cl. A61K 39/00; A61P 3/06; A61P 35/00; C07K 14/47; C07K 14/71; C12N 9/18; C12N 15/86

(52) U.S. Cl. CPC A61K 39/001163; A61K 39/001102; A61K 39/001148; A61P 3/06; A61P 35/00; C07K 14/4703; C07K 14/71; C12N 9/18; C12N 15/86; A61K 2039/53; A61K 2039/575; C12N 2710/10034; C12N 2710/10043; C12N 2710/24034; C12N 2710/24043; C12N 2740/15032; C12N 2740/15043; C12Y 301/01003; C12Y 301/03048

(57) ABSTRACT

Overexpressed proteins associated with inflammatory adipocytes have been demonstrated in TNF-a inflamed adipocyte lines derived from humans and mice as well as in visceral fat derived from mice fed an inflammatory-generating high fat high sucrose diet. The adipocyte-associated proteins are immunogenic in humans and mice, and can be used as a vaccine that drives Type II T-cells to inflammatory adipose tissue. Compositions and methods for the prevention and treatment of disease associated with metabolic obesity, including cancer, such as breast cancer, and non-alcoholic fatty liver disease (NAFLD) are provided.

This application claims benefit of U.S. provisional patent application No. 63/371,999, filed Aug. 19, 2022, the entire contents of which are incorporated by reference into this application.

### ACKNOWLEDGEMENT OF GOVERNMENT SUPPORT

This invention was made with government support under Grant No. W81XWH-11-1-0760, awarded by the U.S. Army Medical Research and Materiel Command. The government has certain rights in the invention.

### REFERENCE TO A SEQUENCE LISTING

The content of the XML file of the sequence listing named “UW78_Seq”, which is 41 kb in size, created on Aug. 3, 2023, and electronically submitted herewith the application, is incorporated herein by reference in its entirety.

### BACKGROUND

Obesity is an important risk factor for breast cancer, especially in postmenopausal women. Obesity occurring after menopause increases the risk of developing estrogen receptor positive breast cancer by 20-40% compared to normal weight women. The dose-response relationship between body mass index (BMI) and postmenopausal cancer consists of a 12% increased risk per 5 kg/m2.1 Obesity is associated with the risk of triple negative breast cancer (TNBC) in pre-menopausal women.2 Individuals with a BMI ≥30 kg/m2 were shown to have an 82% (95% CI 1.32-2.51) increased risk of triple negative breast cancer (TNBC) compared to women with a BMI≤25 kg/m2. Women with metabolic syndrome, the triad of obesity, Type II diabetes, and hypertension, may be at the highest risk.3,4 BMI is not an accurate measure of adiposity. The term “metabolic obesity” is used to describe individuals who are not obese on the basis of height and weight, but who, like people with overt obesity, are hyperinsulinemic, insulin-resistant, and predisposed to type 2 diabetes, hypertriglyceridemia, premature coronary heart disease+ and an increased risk of breast cancer.6-8

There remains a need to treat those with metabolic dysregulation that significantly increases their risk of breast cancer, as well as to effectively target obesity-related conditions, including pre-diabetes, cancers, and inflammation.

### SUMMARY

The material described herein meets these needs and others by providing a vaccine that drives Type II T-cells to inflammatory adipose tissue. Identified herein are overexpressed proteins associated with inflammatory adipocytes, which are immunogenic in humans and mice. Newly identified antigens, DUSP1, FABP4, PAI-1, and ATGL, and epitopes thereof, can be used in such a vaccine, as well as epitopes of IGF-1R and HIF1a identified herein. Thus, provided are, inter alia, a nucleic acid molecule comprising a nucleic acid sequence encoding a polypeptide comprising at least two epitopes selected from the group consisting of: (a) amino acids 53-67 of FABP4; (b) amino acids 29-43 of DUSP1; (c) amino acids 94-108, 120-134, 158-172, 186-200, 270-284 and/or 288-302 of PAI-1; (d) amino acids 93-107, 213-227 and/or 374-388 of ATGL; (e) amino acids 276-295 of HIF1a; (f) amino acids 388-402 and/or 545-558 of IGF-1R; and (g) an amino acid sequence having at least 90% identity with any one of the foregoing. In some embodiments, the polypeptide is selected from the group consisting of: (a) amino acids 53-67 of FABP4; (b) amino acids 29-43 of DUSP1; (c) amino acids 94-108, 120-134, 158-172, 186-200, 270-284 and/or 288-302 of PAI-1; (d) amino acids 93-107, 213-227 and/or 374-388 of ATGL; (e) amino acids 276-295 of HIF1a; (f) amino acids 388-402 and/or 545-558 of IGF-1R; and (g) an amino acid sequence having at least 90% identity with any one of the foregoing. In some embodiments, the polypeptide comprises each of: (a) amino acids 53-67 of FABP4; (b) amino acids 29-43 of DUSP1; (c) amino acids 94-108 and 158-172 of PAI-1; (d) amino acids 213-227 and 374-388 of ATGL; (e) amino acids 276-295 of HIF1a; and (f) amino acids 388-402 and 545-558 of IGF-1R. The amino acid sequences of these epitopes are listed in Table 1.

In some embodiments, the polypeptide is selected from amino acids 53-67 of FABP4, amino acids 29-43 of DUSP1, amino acids 276-295 of HIF1a, and a combination thereof. These epitopes are associated with more IL-10-secreting T cells.

In some embodiments, the polypeptide comprises amino acids 94-108 and/or 158-172 of PAI-1, amino acids 213-227 and/or 374-388 of ATGL, amino acids 388-402 and/or 545-558 of IGF-1R, or a combination thereof. These epitopes are associated with significantly more antigen-specific IL-10 response.

In some embodiments, the polypeptide comprises one or more epitopes of (a), (b), (c), and (d) as listed above. In some embodiments, the polypeptide comprises at least three epitopes selected from (a), (b), (c), and (d). In some embodiments, the polypeptide comprises at least one of each of (a), (b), (c), and (d).

In some embodiments, the polypeptide comprises at least one of each of (a), (b), (c), (d), (e), and (f), and optionally further comprises one or more linker sequences disposed between the epitopes. In some embodiments, the linker sequence is GPGPG or GGGS.

In some embodiments, the nucleic acid sequence comprises a promoter sequence. In some embodiments, the nucleic acid sequence comprises a heterologous sequence. In some embodiments, the heterologous sequence encodes a promoter, a transcriptional start site, a translational start site, a detectable marker, a mRNA processing splice site, a polyadenylation sequence, and/or a regulatory element.

Also described herein is a vector comprising the nucleic acid molecule as described herein, wherein the vector is capable of directing expression of the encoded polypeptide(s). In some embodiments, the vector is a DNA plasmid, messenger RNA (mRNA), or viral vector. In some embodiments, the viral vector is a lentiviral vector, an adenoviral vector, or a poxviral vector. In some embodiments, the vector is mRNA.

Additionally provided herein is a polypeptide comprising at least two epitopes selected from the group consisting of the epitopes listed in Table 1. In some embodiments, the epitopes selected include an amino acid sequence having at least 90% identity with any one of the amino acid sequences listed in Table 1. In some embodiments, the polypeptide comprises a combination of at least three, four, five, six, or more of the epitopes listed in Table 1. In some embodiments, the polypeptide comprises at least one of each of (a), (b), (c), (d), (e), and (f). In some embodiments, the polypeptide further comprises one or more linker sequences disposed between the epitopes.

Also provided is a composition comprising the polypeptide described herein. In some embodiments, the composition further comprises an adjuvant.

Additionally, described herein is a method for eliciting a Type 2 immune response directed at inflammation related to obesity, for preventing or treating inflammation in obesity, and/or for preventing or treating disease associated with metabolic obesity in a subject. In some embodiments, the method comprises administering to the subject a composition comprising a nucleic acid molecule, polypeptide, or composition described herein. In some embodiments, the disease associated with metabolic obesity is cancer. In some embodiments, the cancer is breast cancer. In some embodiments, the disease associated with metabolic obesity is non-alcoholic fatty liver disease (NAFLD), metabolic-associated fatty liver disease (MAFLD), or pre-diabetes.

In some embodiments, the subject is human. In some embodiments, the subject is suspected of having, or has been diagnosed with obesity, a disease associated with metabolic obesity, cancer, such as pre-diabetes/insulin insensitivity, breast cancer, MAFLD, or NAFLD.

### BRIEF DESCRIPTION OF THE DRAWINGS

FIGS. 1A-1D. IL-10-selective epitopes can be identified from aberrantly expressed proteins in adipose tissue in obesity. Th ratio when stimulated with the indicated epitope from (1A) FABP4, (1B) DUSP1 (1C) ATGL or (1D) PAI1. Bars right of “0” denote the Th-2 selective epitopes, and bars left of “0” denote the Th1 selective epitopes. n=10 healthy donors.

FIGS. 2A-2D. Overexpressed antigens identified in human obese adipose tissue are also overexpressed in the adipose tissue of obese mice. (2A) Representative Western blots for the indicated proteins in unstimulated differentiated mouse adipocytes or TNFa-stimulated differentiated mouse adipocytes, ***<0.001, ****p<0.0001. (2B) Relative pixel density (±SD) as measured by densitometry for the indicated proteins. (2C) Representative

Western blot of cell lysate for the indicated antigen from mice fed a high fat diet (HFD) or normal chow diet (NCD). a/B Tubulin was used as a loading control. (2D) Relative pixel density (±SEM) as measured by densitometry normalized to expression observed in the tissue of mice on a NCD; n=5 mice; **p<0.05, **p<0.01, **p<0.001.

FIGS. 3A-3F. IL-10-secreting epitopes identified via in vitro screening generated a Th2-selective response after immunization with each single antigen. Mean (+SEM) IL-10 corrected spots per well (CSPW) in mice vaccinated with a peptide or peptide pool and splenocytes stimulated with the indicated epitope from (3A) FABP4, (3B) DUSP1, (3C) HIF1a, (3D) PAI1, (3E) ATGL and (3F) a IGF-IR presented as box plots showing all points from each individual mice. n=8-12 mice/group; **p<0.01, ***p<0.001, ****p<0.0001.

FIGS. 4A-4C. AdVac immunization generated an IL-10 dominant T-cell response to all antigens in the majority of mice. (4A) Corrected IL-10 spots per well (cSPW) in mice immunized with AdVac and splenocytes stimulated with the indicated antigen presented as box and whisker plots, line at median and whiskers minimum to maximum, showing all points. (4B) Percent of mice responding to the indicated antigen. (4C) Percent of mice responding to zero antigens (bottom bar), three antigens (white bar, second from bottom), five antigens (middle bar) or six antigens (uppermost bar) n=10 mice/group; *p<0.05, **p<0.01.

FIG. 5. Experimental design for both the male C57BL/6 diet induced obesity model and the female TgMMTV-neu mouse model of mammary cancer. wks=weeks of age.

FIGS. 6A-6D. AdVac increased glucose sensitivity and reduced insulin resistance in obese mice. (6A) Glucose tolerance test (GTT) in lean mice (triangles) or obese mice immunized with adjuvant alone (Control; solid circles) or AdVac (open squares). (6B) Area under the curve (AUC) for the GTT. (6C) Insulin tolerance test (ITT) in lean mice (triangles) or obese mice immunized with control (solid circles) or the AdVac (open squares). (6D) AUC for ITT. n=20 mice/group; **p<0.01, ***p<0.001, ****p<0.0001 (compared to control obese).

FIGS. 7A-7D. AdVac reduced CD8+ T cell and increased T-regulatory cell levels in the adipose tissue of obese mice. Percent CD8+ T-cells in all viable lymphocytes from (7A) adipose tissue or (7B) spleen from lean mice or obese mice immunized with adjuvant alone (Alum) or the AdVac vaccine. Percent CD4+/FOXP3+ T-regulatory cells from (7C) adipose tissue or (7D) spleen from lean mice or obese mice immunized with Alum or AdVac. All data are presented as box and whisker plots, horizontal line at median, whickers minimum to maximum, showing all points; n=17-20 mice/group; *p,0.05, **p<0.01, ****p<0.0001.

FIGS. 8A-8F. ADVac metabolically reprograms the adipose tissue microenvironment. (8A) Heatmap of metabolites in adipose tissue in obese mice immunized with adjuvant alone (Control) or ADVac. (8B) The top four metabolite pathways associated with significantly decreased metabolite levels after ADVac immunization. Normalized metabolite levels for (8C) Glutamine, (8D) Niacinamide, (8E) Glucose-1-phosphate, (8F) Glucose-6-phosphate in obese mice immunized with adjuvant only (control) or ADVac or lean untreated mice presented as box and whisker plots showing all points, horizontal line at median, whiskers minimum to maximum. n=7-10 mice/group; **p<0.01, ***p<0.001, ****p<0.0001.

FIGS. 9A-9B. Obese TgMMTV-neu develop a greater number of tumors with a faster growth rate than lean TgMMTV-neu mice. (9A) Kaplan-Meier curve demonstrating percent tumor free in control obese (lower line) and obese mice immunized with AdVac (upper line). (9B) Tumor growth rate (mm3/day) for lean or obese mice. n=15 mice/group; **p<0.01, ***p<0.001.

FIGS. 10A-10C. AdVac immunization decreases CD8+ T-cells in mammary tissue, lowers serum leptin levels, and results in significant tumor inhibition in obese Tg-MMTV-neu mice. (10A) Percent CD8+ T-cells in viable lymphocytes derived from mammary fat from the indicated treatment group. (10B) Mean (+SEM) serum leptin (ng/ml) from mice immunized with the indicated vaccine. (10C) Kaplan-Meier curve demonstrating percent tumor free in control obese (lower line) and obese mice immunized with AdVac (upper line). n=7-15 mice/group; *p<0.05, **p<0.01, ***p<0.001.

### DETAILED DESCRIPTION

The invention described herein is based on the discovery and identification of overexpressed proteins associated with inflammatory adipocytes. The overexpression of these antigens has been demonstrated in TNF-α inflamed adipocyte lines derived from humans and mice as well as in visceral fat derived from mice fed an inflammatory-generating high fat high sucrose (HFHS) diet. The adipocyte-associated proteins are immunogenic in humans and mice. This discovery provides for the development of compositions and methods for the prevention and treatment of disease associated with metabolic obesity, including cancer, such as breast cancer, and non-alcoholic fatty liver disease (NAFLD).

### DEFINITIONS

All scientific and technical terms used in this application have meanings commonly used in the art unless otherwise specified. As used in this application, the following words or phrases have the meanings specified.

As used herein, the term “comprising” is intended to mean that the compositions and methods include the recited elements, but do not exclude others. As used herein, the transitional phrase “consisting essentially of” (and grammatical variants) is to be interpreted as encompassing the recited materials or steps “and those that do not materially affect the basic and novel characteristic(s)” of the recited embodiment. Thus, the term “consisting essentially of” as used herein should not be interpreted as equivalent to “comprising.” “Consisting of” shall mean excluding more than trace elements of other ingredients and substantial method steps for administering the compositions disclosed herein. Aspects defined by each of these transition terms are within the scope of the disclosure herein.

As used herein, a “linker” refers to an amino acid sequence that connects two proteins, polypeptides, peptides, domains, regions, or motifs and may provide a spacer function compatible with interaction of the two sub-binding domains so that the resulting polypeptide retains a specific binding affinity to a target molecule or retains signaling activity. In certain embodiments, a linker is comprised of about two to about 35 amino acids, for instance, or about four to about 20 amino acids or about eight to about 15 amino acids or about 15 to about 25 amino acids.

As used herein, the terms “nucleic acid sequence” or “polynucleotide” refers to nucleotides of any length which are deoxynucleotides (i.e. DNAs), or derivatives thereof; ribonucleotides (i.e. RNAs) or derivatives thereof; or peptide nucleic acids (PNAs) or derivatives thereof. The terms include, without limitation, single-stranded, double-stranded, or multi-stranded DNA or RNA, genomic DNA, cDNA, DNA-RNA hybrids, oligonucleotides (oligos), or other natural, synthetic, modified, mutated or non-natural forms of DNA or RNA.

The term “vector” refers to, without limitation, a recombinant genetic construct or plasmid or expression construct or expression vector that retains the ability to infect and transduce non-dividing and/or slowly-dividing cells and integrate into the target cell's genome. The vector may be derived from or based on a wild-type virus. Aspects of this disclosure relate to an adeno-associated virus vector, an adenovirus vector, and a lentivirus vector.

The term “expression control element” as used herein refers to any sequence that regulates the expression of a coding sequence, such as a gene. Exemplary expression control elements include but are not limited to promoters, enhancers, microRNAs, post-transcriptional regulatory elements, polyadenylation signal sequences, and introns. Expression control elements may be, without limitation, constitutive, inducible, repressible, or tissue-specific. A “promoter” is a control sequence that is a region of a polynucleotide sequence at which initiation and rate of transcription are controlled. It may contain genetic elements at which regulatory proteins and molecules may bind such as RNA polymerase and other transcription factors. In some embodiments, expression control by a promoter is tissue-specific. An “enhancer” is a region of DNA that can be bound by activating proteins to increase the likelihood or frequency of transcription. Non-limiting exemplary enhancers and posttranscriptional regulatory elements include the CMV enhancer and WPRE.

The term “multicistronic” or “polycistronic” or “bicistronic” or tricistronic” refers to mRNA with multiple, i.e., double or triple coding areas or exons, and as such will have the capability to express from mRNA two or more, or three or more, or four or more, etc., proteins from a single construct. Multicistronic vectors simultaneously express two or more separate proteins from the same mRNA. The two strategies most widely used for constructing multicistronic configurations are through the use of 1) an IRES or 2) a 2A self-cleaving site. An “IRES” refers to an internal ribosome entry site or portion thereof of viral, prokaryotic, or eukaryotic origin which are used within polycistronic vector constructs. In some embodiments, an IRES is an RNA element that allows for translation initiation in a cap-independent manner. The term “self-cleaving peptides” or “sequences encoding self-cleaving peptides” or “2A self-cleaving site” refer to linking sequences which are used within vector constructs to incorporate sites to promote ribosomal skipping and thus to generate two polypeptides from a single promoter, such self-cleaving peptides include without limitation, T2A, and P2A peptides or sequences encoding the self-cleaving peptides.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of disclosed herein.

Percent similarity or percent complementary of any of the disclosed sequences may be determined, for example, by comparing sequence information using the GAP computer program, version 6.0, available from the University of Wisconsin Genetics Computer Group (UWGCG). The GAP program utilizes the alignment method of Needleman and Wunsch (1970). Briefly, the GAP program defines similarity as the number of aligned symbols (i.e., nucleotides or amino acids) which are similar, divided by the total number of symbols in the shorter of the two sequences. The preferred default parameters for the GAP program include: (1) a unary comparison matrix (containing a value of 1 for identities and 0 for non-identities) for nucleotides, and the weighted comparison matrix of Gribskov and Burgess (1986), (2) a penalty of 3.0 for each gap and an additional 0.10 penalty for each symbol in each gap; and (3) no penalty for end gaps.

“Nucleotide sequence” refers to a heteropolymer of deoxyribonucleotides, ribonucleotides, or peptide-nucleic acid sequences that may be assembled from smaller fragments, isolated from larger fragments, or chemically synthesized de novo or partially synthesized by combining shorter oligonucleotide linkers, or from a series of oligonucleotides.

As used herein, the terms “protein”, “peptide”, and “polypeptide” refer to amino acid subunits, amino acid analogs, or peptidomimetics. The subunits may be linked by peptide bonds. In another aspect, the subunit may be linked by other bonds, e.g., ester, ether, etc. As used herein the term “amino acid” refers to either natural and/or unnatural or synthetic amino acids.

As used herein, the term “recombinant expression system” or “recombinant expression vector” refers to a genetic construct for the expression of certain genetic material formed by recombination.

The term “effective amount” or “therapeutically effective amount” or “prophylactically effective amount”, refer to an amount of an active agent described herein that is effective to provide the desired/intended result and/or biological activity. Thus, for example, in various embodiments, an effective amount of a composition described herein is an amount that is effective to result in remission or slowing the progression of disease, and/or to improve or to ameliorate symptoms of and/or to treat disease.

When the disclosure herein relates to a small molecule, polypeptide, protein, polynucleotide, nucleic acid, oligonucleotide, antisense, or miRNA, an equivalent or a biologically equivalent of such is intended within the scope of this disclosure. As used herein, the term “biological equivalent thereof” is intended to be synonymous with “equivalent thereof” when referring to a reference small molecule, polypeptide, protein, polynucleotide, nucleic acid, oligonucleotide, antisense, or miRNA even those reference molecules having minimal homology while still maintaining desired structure or functionality. Unless specifically recited herein, it is contemplated that any nucleic acid, polynucleotide, oligonucleotide, antisense, miRNA, polypeptide, or protein mentioned herein also includes equivalents thereof. For example, an equivalent intends at least about 70% homology or identity, or at least 80% homology or identity and alternatively, or at least about 85%, or alternatively at least about 90%, or alternatively at least about 95%, or alternatively 98% percent homology or identity and exhibits substantially equivalent biological activity to the reference protein, polypeptide or nucleic acid. Alternatively, when referring to polynucleotides, an equivalent thereof is a polynucleotide that hybridizes under stringent conditions to the reference polynucleotide or its complement.

In some embodiments disclosed herein, the polypeptide and/or polynucleotide sequences are provided herein for use in gene and protein transfer and expression techniques described herein. Such sequences provided herein can be used to provide the expression product as well as substantially identical sequences that produce a protein that has the same biological properties. These “biologically equivalent” or “biologically active” or “equivalent” polypeptides are encoded by equivalent polynucleotides as described herein. They may possess at least 60%, or alternatively, at least 65%, or alternatively, at least 70%, or alternatively, at least 75%, or alternatively, at least 80%, or alternatively at least 85%, or alternatively at least 90%, or alternatively at least 95% or alternatively at least 98%, identical primary amino acid sequence to the reference polypeptide when compared using sequence identity methods run under default conditions. Specific polynucleotide or polypeptide sequences are provided as examples of particular embodiments. Modifications may be made to the amino acid sequences by using alternate amino acids that have similar charge.

Additionally, an equivalent polynucleotide is one that hybridizes under stringent conditions to the reference polynucleotide or its complement or in reference to a polypeptide, a polypeptide encoded by a polynucleotide that hybridizes to the reference encoding polynucleotide under stringent conditions or its complementary strand. Alternatively, an equivalent polypeptide or protein is one that is expressed from an equivalent polynucleotide.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PC reaction, or the enzymatic cleavage of a polynucleotide by a ribozyme.

Examples of stringent hybridization conditions include: incubation temperatures of about 25 °C to about 37 °C; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40 °C to about 50 °C; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55 °C to about 68 °C; buffer concentrations of about Ix SSC to about 0.1×SSC; formamide concentrations of about 55% to about 75%; and wash solutions of about Ix SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the compositions, therapy, nucleic acid molecules, and methods disclosed herein for inducing an immune response, beneficial or desired results can include one or more, but are not limited to, alleviation or amelioration of one or more symptoms of disease associated with metabolic obesity, diminishment of same, stabilized (i.e., not worsening) state of the condition (including disease), delay or slowing of the condition (including disease), progression, amelioration or palliation of a condition (including disease), states of and remission of (whether partial or total) disease associated with metabolic obesity, whether detectable or undetectable.

As used herein, the term “isolated” means that a naturally occurring DNA fragment, DNA molecule, coding sequence, or oligonucleotide is removed from its natural environment, or is a synthetic molecule or cloned product. Preferably, the DNA fragment, DNA molecule, coding sequence, or oligonucleotide is purified, i.e., essentially free from any other DNA fragment, DNA molecule, coding sequence, or oligonucleotide and associated cellular products or other impurities.

The term “cell” as used herein refers to either a prokaryotic or eukaryotic cell, optionally obtained from a subject or a commercially available source. Cells treated, transfected, transformed, or otherwise in contact with compositions and/or nucleic acid molecules disclosed herein, include without limitation, cells of a human, non-human animal, mammal, or non-human mammal, including without limitation, cells of murine, canine, or non-human primate species. Cells treated, transfected, transformed, or otherwise in contact with compositions and/or nucleic acid molecules disclosed herein are, without limitation, T cells, antigen-presenting cells, and other suitable host cells.

As used herein, the term “subject” includes any human or non-human animal. The term “non-human animal” includes all vertebrates, e.g., mammals and non-mammals, such as non-human primates, horses, sheep, dogs, cows, pigs, chickens, and other veterinary subjects.

As used herein, “a” or “an” means at least one, unless clearly indicated otherwise.

As used herein, to “prevent” or “protect against” a condition or disease means to hinder, reduce or delay the onset or progression of the condition or disease,

The term “encode” as it is applied to nucleic acid sequences refers to a polynucleotide which is said to “encode” a polypeptide, an mRNA, or an effector RNA if, in its native state or when manipulated by methods well known to those skilled in the art, can be transcribed and/or translated to produce the effector RNA, the mRNA, or an mRNA that can for the polypeptide and/or a fragment thereof. The antisense strand is the complement of such a nucleic acid, and the encoding sequence can be deduced therefrom.

As used herein, the term “expression” or “gene expression” refers to the process by which polynucleotides are transcribed into mRNA and/or the process by which the transcribed mRNA is subsequently translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample; further, the expression level of multiple genes can be determined to establish an expression profile for a particular sample.

As used herein, the term “functional” may be used to modify any molecule, biological, or cellular material to intend that it accomplishes a particular, specified effect.

The term “about,” as used herein when referring to a measurable value such as an amount, level or concentration, for example and without limitation, is meant to encompass variations of 20%, 10%, 5%, 1%, 0.5%, or even 0.1% of the specified amount, or fold differences in levels of a quantifiable comparison with a standard or control or reference material, such as 1-fold, 2-fold, 3-fold, 4-fold . . . 10-fold, 100-fold, etc. of the specified level of comparison.

The terms “acceptable,” “effective,” or “sufficient” when used to describe the selection of any components, ranges, dose forms, etc. disclosed herein intend that said component, range, dose form, etc. is suitable for the disclosed purpose.

The term “adeno-associated virus” or “AAV” as used herein refers to a member of the class of viruses associated with this name and belonging to the genus dependoparvovirus, family Parvoviridae. Multiple serotypes of this virus are known to be suitable for gene delivery; all known serotypes can infect cells from various tissue types. At least 11 or 12, sequentially numbered, are disclosed in the prior art. Non-limiting exemplary serotypes useful in the methods disclosed herein include any of the 11 or 12 serotypes, e.g., AAV2, AAV5, and AAV8, or variant serotypes, e.g. AAV-DJ. The AAV structural particle is composed of 60 protein molecules made up of VP1, VP2, and VP3. Each particle contains approximately 5 VP1 proteins, 5 VP2 proteins and 50 VP3 proteins ordered into an icosahedral structure.

### ABBREVIATIONS

| | |
|---|---|
| 2-DG: 2-deoxy glucose | HIF1a: Hypoxia-inducible factor |
| | 1-alpha |
| AdVac: adipocyte directed vaccine | HLA: human leukocyte antigen |
| ATGL: Adipose triglyceride lipase | IGF-1R (or IGF1R): Insulin like |
| AUC: area under the curve | growth factor1 receptor |
| BMI: body mass index | ITT: insulin tolerance test |
| C3T: mammary mouse tumor model | MAC: Mexican American Cohort |
| CI: confidence interval | MHC: major histocompatibility |
| | complex |
| CyTOF: cytometry by time of flight | OCR: oxygen consumption rate |
| DCIS: ductal carcinoma in situ | ORO: Oil-Red-O |
| DUSP1: Dual Specificity | PAI1: Plasminogen activator |
| Phosphatase 1 | inhibitor-1 |
| ECAR: extracellular acidification rate | TCR: T-cell receptor |
| ER: estrogen receptor | Th1: T helper 1 cell |
| FABP4: Fatty acid-binding protein 4 | Th2: T helper 2 cell |
| GTT: glucose tolerance | TIL: tumor infiltrating |
| | lymphocytes |
| H&E: hematoxylin and eosin | TNBC: triple negative breast |
| | cancer |
| HFHS: high fat high sucrose | Treg: T-regulatory cell |

### Compositions and Nucleic Acid Molecules

Provided are compositions and/or nucleic acid molecules for preventing and treating inflammation associated with obesity, or a disease associated with metabolic obesity, in a subject. Disclosed herein is a nucleic acid molecule comprising a nucleic acid sequence encoding a polypeptide that includes one or more of the epitopes described herein. In some embodiments, the polypeptide is selected from the group consisting of: (a) amino acids 53-67 of FABP4; (b) amino acids 29-43 of DUSP1; (c) amino acids 94-108, 120-134, 158-172, 186-200, 270-284 and/or 288-302 of PAI-1; (d) amino acids 93-107, 213-227 and/or 374-388 of ATGL; (e) amino acids 276-295 of HIF1a; (f) amino acids 388-402 and/or 545-558 of IGF-1R; and (g) an amino acid sequence having at least 90% identity with any one of the foregoing. In some embodiments, the polypeptide comprises each of: (a) amino acids 53-67 of FABP4; (b) amino acids 29-43 of DUSP1; (c) amino acids 94-108 and 158-172 of PAI-1; (d) amino acids 213-227 and 374-388 of ATGL; (e) amino acids 276-295 of HIF1a; and (f) amino acids 388-402 and 545-558 of IGF-1R. The amino acid sequences of these epitopes are listed in Table 1.

TABLE 1
Epitopes Included in ADVac (indicated) and Candidates for Inclusion in ADVac

| Epitope | Sequence | | |
|---|---|---|---|
| HIF1A-p276-295 | YEYYHALDSDHLTKTHHDMF | SEQ ID NO: 1 | ADVac |
| IGF1R-p388-402 | YSFYVLDNQNLQQLW | SEQ ID NO: 2 | ADVac |
| IGF1R-p545-558* | TQYAVYVKAVTLTMV | SEQ ID NO: 3 | ADVac |
| FABP4 p53-67 | KSESTFKNTEISFIL | SEQ ID NO: 4 | ADVac |
| PAI1-p94-108 | MAPALRHLYKELMGP | SEQ ID NO: 5 | ADVac |
| PAI1-p158-172 | IINDWVKTHTKGMIS | SEQ ID NO: 6 | ADVac |
| DUSP1-p29-43 | CRSFFAFNAGHIAGS | SEQ ID NO: 7 | ADVac |
| ATGL-p213-227 | QFNLRNLYRISKALF | SEQ ID NO: 8 | ADVac |
| ATGL-p374-388 | CQYLVMRAKRKLGRH | SEQ ID NO: 9 | ADVac |

Candidate Epitopes for Inclusion in ADVac

| | | |
|---|---|---|
| ATGL-p93-107 | IIRSFLLKVLPADSH | SEQ ID NO: 10 |
| HIF1a-p312-331 | GGYVWVETQATVIYNTKNSQ | SEQ ID NO: 11 |
| PAI1-288-302 | NMTRLPRLLVLPKFS | SEQ ID NO: 12 |
| PAI1-270-284 | LSALTNILSAQLISH | SEQ ID NO: 13 |
| PAI1-186-200 | LVLVNALYFNGQWKT | SEQ ID NO: 14 |
| PAI1-120-134 | LSALTNILSAQLISH | SEQ ID NO: 15 |
| BCAT1-95-129 | FEGLKAFRGVDNKIRLFQPNLNMDRMYRSAVRATL | SEQ ID NO: 16 |
| BCAT1-140-175 | CIQQLVKLDQEWVPYSTSASLYIRPTFIGTEPSLGV | SEQ ID NO: 17 |
| BCAT1-184-218 | FVLLSPVGPYFSSGTFNPVSLWANPKYVRAWKGGT | SEQ ID NO: 18 |
| BCAT1-252-274 | DHQITEVGTMNLFLYWINEDGEE | SEQ ID NO: 19 |
| BCAT1-328-353 | MFGSGTACVVCPVSDILYKGETIHIP | SEQ ID NO: 20 |
| NDUFA6-p24-44 | FGKMAGSGVRQATSTASTFVK | SEQ ID NO: 21 |
| NDUFA6-p63-89 | RAWYREVPNTVHQFQLDITVKMGRDKV | SEQ ID NO: 22 |
| NDUFA6-p115-140 | ELEETIKVWKORTHVMRFFHETEAPRPKD | SEQ ID NO: 23 |
| GNPDA2-p52-80 | LIEYHKNGHLSFKYVKTFNMDEYVGLPRN | SEQ ID NO: 24 |
| GNPDA2-p93-110 | FFKHIDIDPNNAHILDGN | SEQ ID NO: 25 |
| GNPDA2-p197-224 | REVMILITGAHKAFALYKAIEEGVNHMW | SEQ ID NO: 26 |
| GNPDA2-p248-269 | RVKTVKYFKGLMHVHNKLVDPL | SEQ ID NO: 27 |
| NEGR1-p214-232 | VRKVKVVVNFAPTIQEIKS | SEQ ID NO: 28 |
| NEGR1-p257-286 | WYKGEKKLFNGQQGIIIQNFSTRSILTVTN | SEQ ID NO: 29 |
| NEGR1-p321-353 | GITGSADVLFSCWYLVLTLSSFTSIFYLKNAIL | SEQ ID NO: 30 |
| FMO3-p90-118 | YILAFAKEKNLLKYIQFKTFVSSVNKHPD | SEQ ID NO: 31 |
| FMO3-p139-174 | VFDAVMVCSGHHVYPNLPKESFPGLNHFKGKCFHSR | SEQ ID NO: 32 |
| FMO3-p299-319 | VKPNVKEFTETSAIFEDGTIF | SEQ ID NO: 33 |
| GCK-p185-214 | LRDAIKRRGDFEMDVVAMVNDTVATMISCY | SEQ ID NO: 34 |
| GCK-P295-319 | GKYMGELVRLVLLRLVDENLLFHGE | SEQ ID NO: 35 |
| GCK-p344-362 | DRKQIYNILSTLGLRPSTT | SEQ ID NO: 36 |
| GCK-p413-434 | YKLHPSFKERFASVRRLTPSC | SEQ ID NO: 37 |
| PELI1-p20-57 | LIVLGYNGSLPNGDRGRRKSRFALFKRPKANGVKPSTV | SEQ ID NO: 38 |
| PELI1-p75-102 | HSISYTLSRAQTVVVEYTHDSNTDMFQI | SEQ ID NO: 39 |
| PELI1-p151-179 | ARIYAAGFDSSKNIFLGEKAAKWKTSDGQ | SEQ ID NO: 40 |
| PELI1-p260-284 | SHTPTVKHLEALRQEINAARPQCPV | SEQ ID NO: 41 |
| UBE2D3-p80-135 | SNGSICLDILASQWSPALTISKVLLSICSLLCDPNPDD | SEQ ID NO: 42 |
| | PLVPEIARIYKTDRDKYN | |
| TLE3-p29-52 | IKDEFQFLQAQYHSLKVEYDKLAN | SEQ ID NO: 43 |
| TLE3-p82-105 | AKRLNTILAQIMPFLSQEHQQVA | SEQ ID NO: 44 |
| TLE3-p355-376 | RTPISITSSYAAPFAMMSHHEM | SEQ ID NO: 45 |
| TLE3-p561-588 | PTPRIKAELTSSAPACYALAISPDAKVC | SEQ ID NO: 46 |

*The mature IGF1R protein is cleaved at 30 amino acids at the N-terminus. These amino acid residues are numbered 575-588 when referencing the pro-protein rather than the mature protein sequence.

In some embodiments, the nucleic acid sequence encodes a polypeptide that comprises a combination of one or more epitopes. In some embodiments, the encoded polypeptide comprises one or more of the epitopes listed in Table 1. In some embodiments, the polypeptide comprises the first nine epitopes listed in Table 1. In some embodiments, the polypeptide comprises one or more of the epitopes listed in the latter portion of Table 1 as candidates for inclusion in an ADVac vaccine. In some embodiments, the polypeptide comprises 3, 4, 5, 6, 7, 8, 9, 10, 11, 12, or more of the epitopes listed in Table 1.

10 In some embodiments, the nucleic acid sequence optionally further comprises one or more linker sequences disposed between the epitopes. In some embodiments, the linker sequence is glycine-serine. In some embodiments, the nucleic acid sequence comprises a promoter sequence. In some embodiments, the nucleic acid sequence comprises a heterologous sequence. In some embodiments, the heterologous sequence encodes a promoter, a transcriptional start site, a translational start site, a detectable marker, a mRNA processing splice site, a polyadenylation sequence, and/or a regulatory element.

Such nucleic acid molecules may be delivered by viral or non-viral means. One example of viral delivery is adeno-associated virus (AAV). Other examples include retrovirus, lentivirus, and baculovirus delivery. One example of a non-viral method of delivery is cell penetrating peptide (CPP). Polynucleotide constructs may also be modified, such as through chemical modification, to improve their stability and/or suitability for delivery. In some embodiments, the oligonucleotide is modified by locked nucleic acids and/or phosphorothioate linkages. In some embodiments, a delivery system is selected for improved bioavailability, such as PEGylated liposomes, lipidoids, or biodegradable polymers, as examples.

### Viral Vectors

In some embodiments, the vector disclosed herein is a viral vector. In some embodiments, the vector is an adenoviral vector, an adeno-associated viral (AAV) vector, or a lentiviral vector. In some embodiments, the vector is a retroviral vector, an adenoviral/retroviral chimera vector, a herpes simplex viral I or II vector, a parvoviral vector, a reticuloendotheliosis viral vector, a polioviral vector, a papillomaviral vector, a vaccinia viral vector, or any hybrid or chimeric vector incorporating favorable aspects of two or more viral vectors. In some embodiments, the vector further comprises one or more expression control elements operably linked to the polynucleotide. In some embodiments, the vector further comprises one or more selectable markers.

In some embodiments, the vector disclosed herein is an AAV vector with low toxicity. In some embodiments, the AAV vector does not incorporate into the host genome, thereby having a low probability of causing insertional mutagenesis. In some embodiments, the AAV vector can encode a range total polynucleotides from 4.5 kb to 4.75 kb. In some embodiments, exemplary AAV vectors that may be used in any of the herein described compositions, systems, methods, and kits can include an AAV1 vector, a modified AAV1 vector, an AAV2 vector, a modified AAV2 vector, an AAV3 vector, a modified AAV3 vector, an AAV4 vector, a modified AAV4 vector, an AAV5 vector, a modified AAV5 vector, an AAV6 vector, a modified AAV6 vector, an AAV7 vector, a modified AAV7 vector, an AAV8 vector, an AAV9 vector, an AAV.rh10 vector, a modified AAV.rh10 vector, an AAV.rh32/33 vector, a modified AAV.rh32/33 vector, an AAV.rh43 vector, a modified AAV.rh43 vector, an AAV.rh64R1 vector, and a modified AAV.rh64R1 vector and any combinations or equivalents thereof.

In some embodiments, the vector disclosed herein is a lentiviral vector. In one embodiment, the lentiviral vector is an integrase-competent lentiviral vector (ICLV). In some embodiments, the lentiviral vector can refer to the transgene plasmid vector as well as the transgene plasmid vector in conjunction with related plasmids (e.g., a packaging plasmid, a rev expressing plasmid, an envelope plasmid) as well as a lentiviral-based particle capable of introducing exogenous nucleic acid into a cell through a viral or viral-like entry mechanism. Lentiviral vectors are well-known in the art. In some embodiments, exemplary lentiviral vectors that may be used in relation to any of the herein described compositions, nucleic acid molecules and/or methods, and can include a human immunodeficiency virus (HIV) 1 vector, a modified human immunodeficiency virus (HIV) 1 vector, a human immunodeficiency virus (HIV) 2 vector, a modified human immunodeficiency virus (HIV) 2 vector, a sooty mangabey simian immunodeficiency virus (SIVsm) vector, a modified sooty mangabey simian immunodeficiency virus (SIVsm) vector, a African green monkey simian immunodeficiency virus (SIVAGM) vector, a modified African green monkey simian immunodeficiency virus (SIVAGM) vector, a equine infectious anemia virus (EIAV) vector, a modified equine infectious anemia virus (EIAV) vector, a feline immunodeficiency virus (FIV) vector, a modified feline immunodeficiency virus (FIV) vector, a Visna/maedi virus (VNV/VMV) vector, a modified Visna/maedi virus (VNV/VMV) vector, a caprine arthritis-encephalitis virus (CAEV) vector, a modified caprine arthritis-encephalitis virus (CAEV) vector, a bovine immunodeficiency virus (BIV), or a modified bovine immunodeficiency virus (BIV).

In some embodiments of the compositions and/or nucleic acid molecules and/or methods of the disclosure, a vector of the disclosure is a viral vector. In some embodiments, the viral vector comprises a sequence isolated or derived from a retrovirus. In some embodiments, the viral vector comprises a sequence isolated or derived from a lentivirus. In some embodiments, the viral vector comprises a sequence isolated or derived from an adenovirus. In some embodiments, the viral vector comprises a sequence isolated or derived from an adeno-associated virus (AAV). In some embodiments, the viral vector is replication incompetent. In some embodiments, the viral vector is isolated or recombinant. In some embodiments, the viral vector is self-complementary.

In some embodiments of the compositions and/or nucleic acid molecules and/or methods of the disclosure, the viral vector comprises a sequence isolated or derived from an adeno-associated virus (AAV). In some embodiments, the viral vector comprises an inverted terminal repeat sequence or a capsid sequence that is isolated or derived from an AAV of serotype AAV1, AAV2, AAV3, AAV4, AAV5, AAV6, AAV7, AAV8, AAV9, AAV10, AAV11, or AAV12, or the vector and/or components are derived from a synthetic AAV serotype, such as, without limitation, Anc80 AAV (an ancestor of AAV 1, 2, 6, 8 and 9). In some embodiments, the viral vector is replication incompetent. In some embodiments, the viral vector is isolated or recombinant (rAAV). In some embodiments, the viral vector is self-complementary (scAAV).

In some embodiments of the compositions and methods of the disclosure, a vector of the disclosure is a non-viral vector. In some embodiments, the vector comprises or consists of a nanoparticle, a micelle, a liposome or lipoplex, a polymersome, a polyplex or a dendrimer.

In some embodiments, expression vector or viral vector disclosed herein is used to transfect, transform, or come in contact with a cell which is a eukaryotic cell. In some embodiments, the cell is an animal cell. In some embodiments, the cell is a mammalian cell. In some embodiments, the cell is a bovine, murine, feline, equine, porcine, canine, simian, or human cell.

In some embodiments, a cell is a packaging cell or a producer cell for production of a viral particle.

In some embodiments, provided herein are viral particles comprising, consisting of, or consisting essentially of a vector comprising, consisting of, or consisting essentially of a polynucleotide sequence encoding one or more polypeptides as described herein.

In general, methods of packaging genetic material such as RNA or DNA into one or more vectors is well known in the art. For example, the genetic material may be packaged using a packaging vector and cell lines and introduced via traditional recombinant methods.

In some embodiments, the packaging vector may include, but is not limited to retroviral vector, lentiviral vector, adenoviral vector, and adeno-associated viral vector. The packaging vector contains elements and sequences that facilitate the delivery of genetic materials into cells. For example, the retroviral constructs are packaging plasmids comprising at least one retroviral helper DNA sequence derived from a replication-incompetent retroviral genome encoding in trans all virion proteins required to package a replication incompetent retroviral vector, and for producing virion proteins capable of packaging the replication-incompetent retroviral vector at high titer, without the production of replication-competent helper virus. The retroviral DNA sequence lacks the region encoding the native enhancer and/or promoter of the viral 5′ LTR of the virus, and lacks both the psi function sequence responsible for packaging helper genome and the 3′ LTR, but encodes a foreign polyadenylation site, for example the SV40 polyadenylation site, and a foreign enhancer and/or promoter which directs efficient transcription in a cell type where virus production is desired. The retrovirus is a leukemia virus such as a Moloney Murine Leukemia Virus (MMLV), the Human Immunodeficiency Virus (HIV), or the Gibbon Ape Leukemia virus (GALV). The foreign enhancer and promoter may be the human cytomegalovirus (HCMV) immediate early (IE) enhancer and promoter, the enhancer and promoter (U3 region) of the Moloney Murine Sarcoma Virus (MMSV), the U3 region of Rous Sarcoma Virus (RSV), the U3 region of Spleen Focus Forming Virus (SFFV), or the HCMV IE enhancer joined to the native Moloney Murine Leukemia Virus (MMLV) promoter.

The retroviral packaging plasmid may consist of two retroviral helper DNA sequences encoded by plasmid-based expression vectors, for example where a first helper sequence contains a cDNA encoding the gag and pol proteins of ecotropic MMLV or GALV and a second helper sequence contains a cDNA encoding the env protein. The Env gene, which determines the host range, may be derived from the genes encoding xenotropic, amphotropic, ecotropic, polytropic (mink focus forming) or 10A1 murine leukemia virus env proteins, or the Gibbon Ape Leukemia Virus (GALV env protein, the Human Immunodeficiency Virus env (gp160) protein, the Vesicular Stomatitus Virus (VSV) G protein, the Human T cell leukemia (HTLV) type I and II env gene products, chimeric envelope gene derived from combinations of one or more of the above env genes or chimeric envelope genes encoding the cytoplasmic and transmembrane of the above env gene products and a monoclonal antibody directed against a specific surface molecule on a desired target cell. Similar vector-based systems may employ other vectors such as sleeping beauty vectors or transposon elements.

The resulting packaged expression systems may then be introduced via an appropriate route of administration, discussed in detail with respect to the method aspects disclosed herein. Polypeptides

Also provided is a polypeptide that includes one or more epitopes of MCPyV. In some embodiments, the polypeptide is selected from the group consisting of: (a) amino acids 53-67 of FABP4; (b) amino acids 29-43 of DUSP1; (c) amino acids 94-108, 120-134, 158-172, 186-200, 270-284 and/or 288-302 of PAI-1; (d) amino acids 93-107, 213-227 and/or 374-388 of ATGL; (e) amino acids 276-295 of HIF1a; (f) amino acids 388-402 and/or 545-558 of IGF-1R; and (g) an amino acid sequence having at least 90% identity with any one of the foregoing. The amino acid sequences of these epitopes are listed in Table 1. In some embodiments, the epitopes selected include an amino acid sequence having at least 90% identity with any one of the amino acid sequences listed in Table 1. In some embodiments, the polypeptide comprises a combination of at least three, four, five, six, or more of the epitopes listed in Table 1. In some embodiments, the polypeptide comprises at least one of each of (a), (b), (c), (d), (e), and (f). In some embodiments, the polypeptide further comprises one or more linker sequences disposed between the epitopes.

Pharmaceutical Compositions

Pharmaceutical compositions disclosed herein include one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the disclosure may be formulated for administration as a vaccine, for example, with the inclusion of an adjuvant.

For example, in some embodiments, provided is a composition comprising the polypeptide, wherein the composition further comprises an adjuvant. In some embodiments, the adjuvant elicits T cell responses. In some embodiments, the composition comprises a nucleic acid molecule disclosed herein.

### Methods

Described herein are methods for eliciting a Type 2 immune response directed at inflammation related to obesity, for preventing and treating inflammation in obesity, and for preventing and treating disease associated with metabolic obesity in a subject. Also provided are methods for reducing insulin resistance, reducing systemic leptin levels, and reducing tumors associated with obesity. Examples of disease associated with metabolic obesity include, but are not limited to, diabetes, pre-diabetes, non-alcoholic fatty liver disease (NAFLD), metabolic-associated fatty liver disease (MAFLD), colitis, and cancer, including, for example, breast cancer. In a typical embodiment, the method comprises administering to a subject in need thereof a composition as described herein. Such compositions are formulated to deliver a therapeutically effective amount of the epitopes, as described herein. Determination of amounts and appropriate means of administration are determined under the guidance of a treating physician, taking into account an individual patient's condition.

### Administration and Dosage

The compositions and/or nucleic acid molecules disclosed herein are administered in any suitable manner, often with pharmaceutically acceptable carriers. Suitable methods of administering compositions, compounds, molecules, nucleic acids, and vectors in the context of the present invention to a subject are available, and, although more than one route can be used to administer a particular composition, a particular route can often provide a more immediate and more effective reaction than another route.

The dose administered to a patient, in the context of the disclosure herein, should be sufficient to result in a beneficial therapeutic response in the patient over time, or to inhibit disease progression. Thus, the composition is administered to a subject in an amount sufficient to elicit an effective response and/or to alleviate, reduce, cure or at least partially arrest symptoms and/or complications from the disease. An amount adequate to accomplish this is defined as a “therapeutically effective dose.”

Routes, order and/or frequency of administration of the therapeutic compositions disclosed herein, as well as dosage, will vary from individual to individual, and may be readily established using standard techniques. In general, an appropriate dosage and treatment regimen provides the active compound(s) in an amount sufficient to provide therapeutic and/or prophylactic benefit. Such a response can be monitored by establishing an improved clinical outcome in treated patients as compared to non-treated patients.

### EXAMPLES

The following examples are presented to illustrate the present invention and to assist one of ordinary skill in making and using the same. The examples are not intended in any way to otherwise limit the scope of the invention.

### Example 1: Type I Adipose Inflammation Reflects an Adaptive Immune Response Driving Metabolic Dysregulation

The inflammatory infiltrate found in adipose tissue of “metabolically obese” individuals is an adaptive Type I T-cell mediated response that drives metabolic dysfunction.¹ Infiltration of CD8 T-cells into fat is an early event.² Type I cytokines secreted by CD8 T-cells upregulate costimulatory molecules on enlarged adipocytes which, as antigen presenting cells, provide antigenic peptides to CD4 and CD8 T-cells, further stimulating Type I T-cell activation.³⁻⁵ Thus, inflamed adipocytes vaccine against adipocyte associated proteins. Type I CD4 T-cells become essential to maintaining inflammation.^{6.7} The resulting effector and memory T-cells generated compete for glucose and fatty acids which leads to metabolic dysfunction in both the adipose tissue and the T-cells themselves.⁸ T-cells are not able to maintain immune surveillance® and secretion of adipokines promotes epithelial to mesenchymal and malignant transformations.^{10.11} Losing weight does not solve the problem. Immunologic memory prevents inflammation from resolving even if an individual becomes normal weight. Studies in formerly obese mice show that serum cytokine expression and proinflammatory genes in the mammary fat pad are similar to those in currently obese mice and much higher than in lean controls.^{12,13} Further, T-cell metabolic defects are not fully corrected with weight loss.¹⁴ Similar effects have been observed in humans. While insulin sensitivity is restored after bariatric surgery and weight loss in formerly obese individuals, serum biomarkers of systemic inflammation and pro-inflammatory gene expression in adipose tissue remain^{15,16} and levels of T-cells are unchanged.¹⁷ Obesogenic T-cell memory will confer long-term increases in adipose inflammation in formerly obese individuals.^{18,19,20} Clinical strategies that either lower Type I T-cells or increase Type II (anti-inflammatory) T-cells in inflammatory adipose would have benefit in preventing breast cancer.

### REFERENCES

1. Mathis, D. Cell Metab 17, 851-859 (2013).
2. Nishimura, S., et al. Nat Med 15, 914-920 (2009).
3. Lumeng, C. N., Bodzin, J. L. & Saltiel, A. R. J Clin Invest 117, 175-184 (2007).
4. Xiao, L., et al. Int J Obes (Lond) 40, 112-120 (2016).
5. Missiou, A., et al. Thromb Haemost 103, 788-796 (2010).
6. Deng, T., et al. Cell Metab 17, 411-422 (2013).
7. Cho, K. W., et al. Cell Rep 9, 605-617 (2014).
8. Ringel, A. E., et al. Cell 183, 1848-1866 e1826 (2020).
9. Zhang, C., et al. Cell Metab 31, 148-161 e145 (2020).
10. Avtanski, D., et al. Cytokine 120, 155-164 (2019).
11. Tiwari, P., et al. J Exp Med 216, 1345-1358 (2019).
12. Rossi, E. L., et al. Cancer Prev Res (Phila) 9, 339-348 (2016).
13. Zamarron, B. F., et al. Diabetes 66, 392-406 (2017).
14. Rebeles, J., et al. J Infect Dis 219, 1652-1661 (2019).
15. Hagman, D. K., et al. Metabolism 70, 12-22 (2017).
16. Schmitz, J., et al. Mol Metab 5, 328-339 (2016).
17. Garcia-Rubio, J., et al. Sci Rep 8, 15203 (2018).
18. Zou, J., et al. Cell Mol Immunol 15, 630-639 (2018).
19. Sbierski-Kind, J., et al. J Immunol 205, 45-55 (2020).
20. Cecil, D. L., et al. Breast Cancer Res Treat 139, 657-665 (2013).

### Example 2: Development of a Vaccine that Drives Type II T-Cells to Inflammatory Adipose Tissue

This Example demonstrates the development of a vaccine that can drive Type II T-cells to inflammatory adipose tissue. We have explored the potential of comprehensive metabolomics profiling and have applied an artificial intelligence approach to a large set of plasma mass spectrometry data collected from 353 breast cancer cases and 141 controls at MD Anderson Cancer Center which revealed a signature involving lipids and other metabolites discriminating cancer cases from controls. Validation of the signature in an independent set of 85 cases and 170 matched controls resulted in an area under the curve (AUC) of 0.812 (CI 0.755-0.870). These and our published data related to breast cancer signatures in circulation justify a discovery strategy focused on breast cancer risk markers associated with obesity.

Antigen identification. Antigens were identified by searching on key words: [(“obese” OR “fat” OR “obesity) AND (“inflammation” OR “inflammatory” OR “immune” OR “immunogenic”) AND (“overexpression”) NOT (“macrophage” OR “neutrophil” OR “innate” OR “monocyte” OR “granulocyte” OR “myeloid” OR “dendritic”) NOT (“nervous system” OR “endocrine system” OR “muscular system” OR “hepatic”) NOT (“expressed in brown adipose tissue”) in PubMed, retrieving 19 candidates. Targets were prioritized if they were associated with hypoxia and/or metabolic syndrome. The final 10 candidates identified are listed in Table 3.

Human subjects. Sample collection was approved by the University of Washington (UW) Human Subjects Division. Peripheral blood mononuclear cells (PBMC) from 10 volunteer controls (4 males and 6 females, median age: 49, range 18-79 years) were collected and cryopreserved as previously described (1).

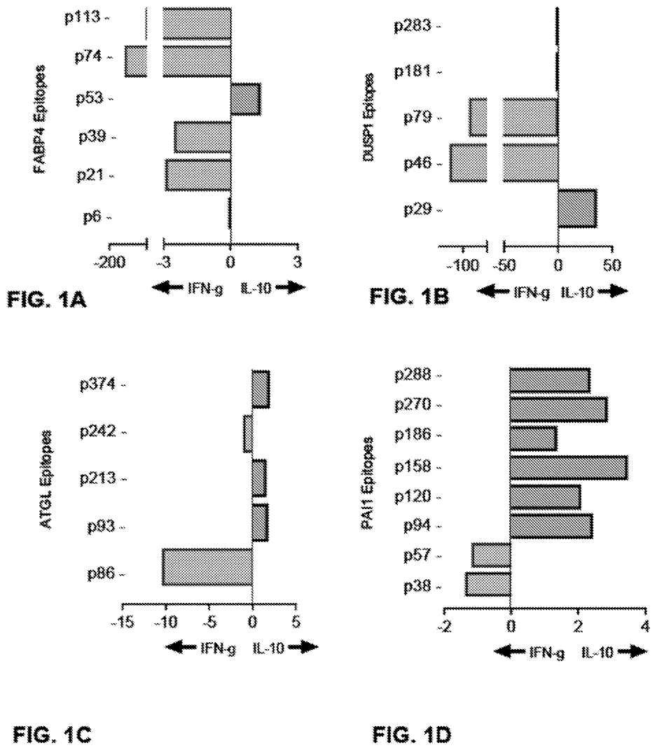

Analysis of peptide-specific T-cell responses. Peptides, predicted to promiscuously bind human MHCII, were selected using web based algorithms as previously described (Table 3) (2). The peptides were constructed and purified by high-performance liquid chromatography (>90% pure; CPC Scientific). Human PBMC were evaluated by ELISPOT for antigen-specific IFN-gamma (γ) or IL-10 production as previously described (3) using 10 μg/ml peptides. Mouse splenic cells were evaluated by ELISPOT for antigen-specific cytokine secretion as published, with the following modifications; splenic cells were incubated with antigens for 72 hr and spots were developed with the AEC substrate kit (BD Biosciences) (2). Positive responses were defined by a statistically significant difference (p<0.05) between the mean number of spots in the experimental wells and the mean number from no antigen control wells, all performed with 4 replicates. Data are reported as the mean number of spots for each experimental antigen minus the mean number of spots detected in no antigen control wells #SEM or SD (corrected spots per well: CSPW per 2×10⁶ PBMC or 3.5×10⁵ splenic cells). The ratio of the product of the mean magnitude and percent incidence of response was calculated from the IFN-γ and IL-10 ELISPOT assays (IL-10^{incidence×magnitude}/IFN-γ^{incidence×magnitude}) (4). Ratios greater than 1.4 and with ≤10% donors generating an IFN-γ response defined Th2-selective epitopes and ratios less than 1 defined Th1-selective epitopes. For ease in a graphic representation of the ratio value of Th1 epitopes, the ratio was reversed (IFN-γ/IL-10) and this value was plotted in FIG. 1.

Animal model. Animal care and use were in accordance with institutional guidelines. Male, C57BL/6 (Jackson Laboratory) were used in this study. Mice were fed a high fat diet (60% fat calories; Bio-serve) or normal chow from 10 weeks old. Mouse weight was measured once per week for the duration of the study. Ten week old TgMMTV-neu mice were fed a HFHS diet for 4 weeks, then randomized into 2 cohorts, one receiving the adjuvant only (Alum) and one receiving ADVac. Mice were sacrificed at 31 weeks of age or when tumor was >1000 mm3.

Metabolic studies. Mice were fasted 4 h prior to the metabolic studies. For the glucose tolerance test, mice were injected with 1 g glucose per kg body weight and for the insulin tolerance test mice, mice were injected with 0.75 U per kg body weight human insulin (Eli Lilly). Glucose concentration was measured on a glucometer (Freestyle Freedom Lite) from blood collected pre-injection and at 30, 60 and 120 min after injection.

Lipidomics. Ten mg of frozen adipose samples were homogenized in 15% water, 60% methanol, and 25% dichloromethane. The mixture was vortexed for 5 s and 25 μL of the isotope labeled internal standards mixture were added to the tube. Samples were left to incubate at room temperature for 30 min. Next, another 55% water and 45% dichloromethane were added to the tube, followed by gentle vortexing for 5 s, and centrifugation at 2500×g at 15 °C for 10 min. The bottom organic layer was transferred to a new tube and 900 μL of dichloromethane was added to the original tube for a second extraction. The combined extracts were concentrated under nitrogen and reconstituted in 250 μL of the running solution (10 mM ammonium acetate in 50:50 methanol:dichloromethane). Quantitative lipidomics was performed with the Sciex Lipidyzer platform consisting of Shimadzu Nexera X2 LC-30AD pumps, a Shimadzu Nexera X2 SIL-30AC autosampler, and a Sciex QTRAP® 5500 mass spectrometer equipped with SelexION® for differential mobility spectrometry (DMS). 1-propanol was used as the chemical modifier for the DMS. Samples were introduced to the mass spectrometer by flow injection analysis at 8 μL/min. Each sample was injected twice, once with the DMS on (PC/PE/LPC/LPE/SM), and once with the DMS off (CE/CER/DAG/DCER/FFA/HCER/LCER/TAG).

The lipid molecular species were measured using multiple reaction monitoring (MRM) and positive/negative polarity switching. Positive ion mode detected lipid classes SM/DAG/CE/CER/DCER/HCER/DCER/TAG and negative ion mode detected lipid classes LPE/LPC/PC/PE/FFA. A total of 1070 lipids and fatty acids were targeted in the analysis. Data were acquired and processed using Analyst 1.6.3 and Lipidomics Workflow Manager 1.0.5.0.

Aqueous metabolomics. Ten mg of frozen adipose tissue was homogenized in 80% Methanol: 9% PBS: 11% H2O. After centrifugation, aqueous extracts were collected and dried at 30 °C in a Speed-Vac. All samples were analyzed using an Agilent 7890 GC instrument equipped with a 5977 mass selective detector (MSD), employing an HP-5 MS UI GC column. GCMS conditions were used according to the Fiehn library instructions from Agilent. Retention time of individual metabolites were annotated according to known standards. Peak intensities were normalized to total ion count (TIC) and protein concentration and depicted as fold change from control. Data were acquired and processed using MetaboAnalyst 4.0.2.

Isolation of stromal vascular cells and flow cytometry. Epididymal adipose tissue was collected, weighed and put in a C-tube (Miltenyi) containing 5 ml DMEM supplemented with 10 mg/mL BSA, 0.03 mg/ml. Liberase Blendzyme 3 (Worthington) and 50 U/mL DNase, then incubated in the gentleMACs dissociator using the manufacturer's “mTDK1” program. The aqueous slurry was filtered and the red blood cells were lysed. The washed cells (1-2×106) were incubated with a live/dead stain (FVS450; ThermoFisher), washed and incubated with an FC-receptor blocking antibody to reduce non-specific binding. After washing, the cells were stained with fluorochrome-conjugated monoclonal antibodies for phenotyping analyses. The cells were surface-stained with CD3 PE-Cy7, CD4 BV605 and CD8 APC-Cy7. After washing, the cells were stained with FOXP3 Alex 488 according to eBioscience's FOXP3 staining protocol. The % of CD8+ cells were analyzed among viable lymphocytes. FOXP3 positive cells was analyzed among CD3+CD4+ T cells. Only data from samples counting >350 cells are included.

Vaccination. Mice were immunized subcutaneously using a 26 1/2 G needle. Each mouse was injected with 50 μl of a peptide pool (100 μg each) in Imject Alum Adjuvant (Thermo Fisher Scientific). To generate an effective immune response to an overexpressed self-antigen, four immunizations were given two weeks apart.

Cell culture and protein expression, 3T3-L1 pre-adipocytes were differentiated into adipocytes with 0.5 mM IBMX, 1 ug/ml insulin, 0.25 μM dexamethasone and 2 μM rosiglitazone for 10 days. 94% of the culture was positive for oil red-O. Cultures were stimulated with 2.5 nM TNFa daily for 5 days and confirmed to be inflammatory (increased activation of NFKb), insulin resistant (decreased expression of IRS-1) and had a decreased expression of adiponectin. Adipose tissue was collected and homogenized using a mechanical sonicator, and the lipid layer was removed. Cell lysates were separated by SDS/PAGE (4) and Western blot performed. Antibodies used were rabbit anti-mouse IGF-IR (2 μg/mL; Genetex), rabbit anti-mouse ATGL (1 μg/ml; Genetex), rabbit anti-mouse PAI-1 (5 μg/ml; Genetex) goat anti-mouse FABP4 (1 μg/ml; Santa Cruz Biotech), rabbit anti-mouse HIF1a (2 μg/mL; abcam), HRP-conjugated goat anti-rabbit and rabbit anti-goat (diluted 1:10,000; Invitrogen).

Serum ELISA. Measurement of Serum Amyloid A was performed with the SAA Mouse ELISA kit (Invitrogen). Measurement of IL-6 and Leptin was performed with the respective Mouse Duoset ELISAs (Rand D Systems). Cholesterol was measured with the HDL and LDL/VLDL Quantification Kit and triglycerides were measured with the TG Quantification Kit (LS Bio). All ELISAs were performed according to the manufacturer's directions.

Statistical analysis. Data are expressed as mean±standard error of the mean (SEM). Statistical analysis was performed with Prism 7.0 or 8.0 software (GraphPad). Statistical comparisons between 2 groups were conducted by unpaired, two-tailed t-test. Statistical comparisons between 3 or more groups were conducted by one-way ANOVA followed by a Tukey posthoc analysis to determine statistical significance. The value of p<0.05 was considered statistically significant.

TABLE 2
Genes Associated With Obesity, Metabolic
Syndrome, and/or Type 2 Diabetes

| | | importance in obesity/ | |
|---|---|---|---|
| Source | Gene | MetS/T2D | |
| Adipose | NDUFA6 | participates in the | PMID: 34793269 |
| Microarray | | regulation of adipogenic | |
| | | differentiation. | |
| | TLE3 | asscociated with T2D | PMID: 25249007 |
| Lit search | BCAT1 | associated with insulin | PMID: 27642608; |
| | | resistance; enzyme | PMID: 27642608; |
| | | associated with BCAA | PMID: 34932898 |
| | | catabolism, which is | |
| | | associated with increased | |
| | | lipogenesis; SNP associtaed | |
| | | with development of T2D | |
| | GNPDA2 | obesity-related SNPs, | PMID: 19851340; |
| | | regulates Glu homeostasis | PMID: 20215397; |
| | | | PMID: 23375129 |
| | NEGR1 | obesity-related SNPs | PMID: 19851341 |
| NASH | FMO3 | suppressed by insulin in | PMID: 25849138 |
| microarray | | vitro, increased in obese/ | |
| | | insulin-resistant humans. | |
| | | Knockdown prevents the | |
| | | development of | |
| | | hyperglycemia, | |
| | | hyperlipidemia and | |
| | | atherosclerosis. | |
| | GCK | SNPs associated with | PMID: 34746319 |
| | | development of T2D | |
| | PELI1 | mediated ubiquitination in | PMID: 28746869 |
| | | M1 macrophages regulating | |
| | | glucose intolerance in | |
| | | obesity, | |
| | UBE2D3 | associated with gestational | PMID: 26869332 |
| | | diabetes | |

### Results

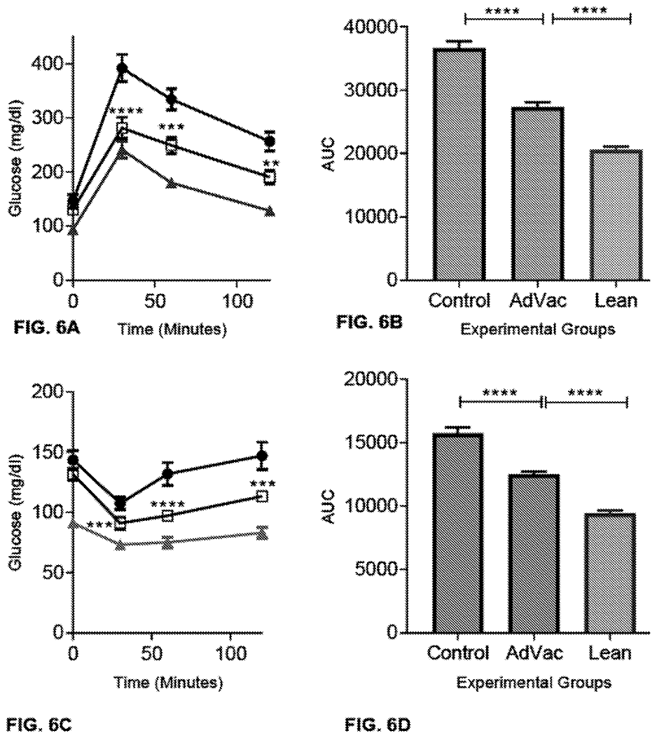

IL-10-selective epitopes can be identified from aberrantly expressed proteins in adipose tissue in obesity. We sought to identify candidate antigens as targets for inclusion in a multi-epitope, multi-antigen anti-inflammatory vaccine. IFN-γ and IL-10-inducing epitopes were determined from the candidate antigens (Table 3). We have previous determined IL-10 selective epitopes for IGF-IR (p388-402 and p545-558) and HIF1a (p276-295) (4, 5). One of six epitopes from FABP4 (p53-67; FIG. 1A), 1/5 epitopes in DUSP1 (p29-43; FIG. 1B), 2/5 epitopes from ATGL (p93-107, p213-227 and p374-388; FIG. 1C) and 6/8 epitopes from PAI-1 (p94-108, p120-134, p158-172, p186-200, p270-284 and p288-302) were identified as inducing primarily 5 10 IL-10 in PBMC (FIG. 1D). No Th2-selective epitopes were identified in IGF-1, 11BHSDB1, SEMA3E or SOCS3. These antigens were not considered further.

TABLE 3
Candidate Antigens, Identification Codes and Epitope Locations

| Antigen | Reference code | Epitopes | Antigen | Reference code | Epitopes |
|---|---|---|---|---|---|
| ATGL | NP_065109.1 | p86-100 | IGF1 | NP_000609.1 | p18-32 |
| | | p93-107 | | | p33-47 |
| | | p213-227 | | | p64-78 |
| | | p242-256 | | | p97-111 |
| | | p374-388 | | | p112-126 |
| 11HSDB1 | NP_001193670.1 | p3-17 | PAI1 | NP_000593.1 | p38-52 |
| | | p14-28 | | | p57-71 |
| | | p144-158 | | | p94-108 |
| | | p165-179 | | | p120-134 |
| | | p177-191 | | | p158-172 |
| DUSP1 | NP_004408.1 | p29-43 | | | p186-200 |
| | | p46-60 | | | p270-284 |
| | | p79-93 | | | p288-302 |
| | | p181-195 | SEMA3E | NP_001171600.1 | p197-211 |
| FABP4 | NP_001433.1 | p6-20 | | | p242-256 |
| | | p21-35 | | | p327-341 |
| | | p39-53 | | | p340-352 |
| | | p53-67 | | | p402-416 |
| | | p74-88 | | | p632-646 |
| | | p283-297 | SOCS3 | NP_003946.3 | p29-43 |
| FASN | NP_004095.4 | p113-127 | | | p44-58 |
| | | p54-70 | | | p59-73 |
| | | p127-142 | | | p74-88 |
| | | p143-161 | | | p117-131 |
| | | p162-179 | | | |
| | | p464-478 | | | |
| | | p542-561 | | | |
| | | p1026-1044 | | | |
| | | p1341-1359 | | | |
| | | p1654-1670 | | | |
| | | p1926-1940 | | | |
| | | p2337-2352 | | | |

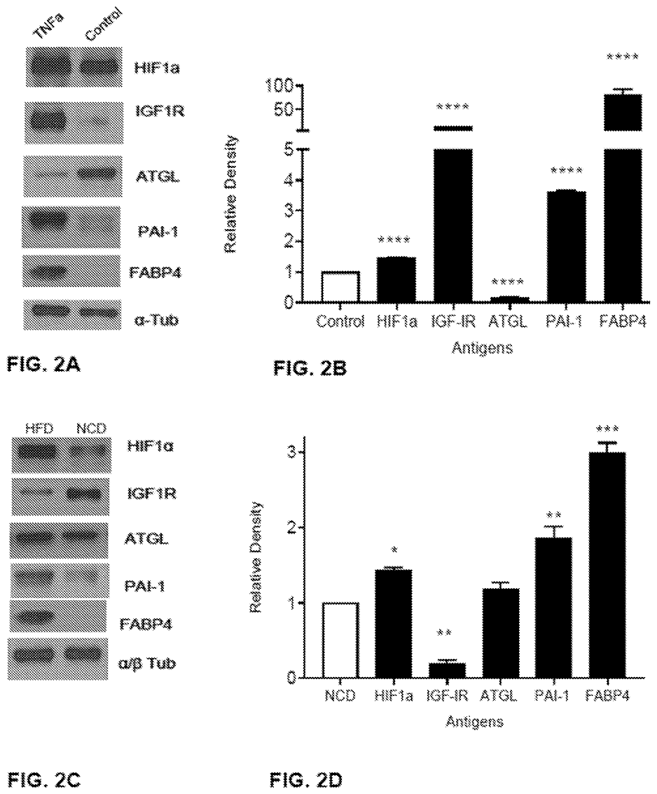

We next questioned whether the candidate antigens were truly upregulated in inflammatory adipocytes. Protein expression of HIF1a, IGF-IR, PAI-1 and FABP4 were upregulated after rmTNF-α stimulation (p<0.0001 for all) compared to resting control cells (FIGS. 2A and 2B). Expression of ATGL was significantly decreased after stimulation compared to control (p<0.0001). We were unable to perform protein expression for DUSP1, but we observed a significant increase in gene expression after stimulation with TNFa as compared to the non-inflammatory control (p<0.0001). In similar experiments, we questioned whether these proteins would also be upregulated in inflammatory fat derived from obese mice, providing us with a model to test adipocyte targeting Th2 selective vaccines. Western blot analysis demonstrated that the majority of candidate antigens evaluated were overexpressed in inflammatory fat as compared to controls (FIGS. 2C and 2D). Western blot analysis revealed increased protein expression of HIF1a (p=0.018), PAI-1 (p<0.0001) and FABP4 (p<0.0001) compared to expression in the adipose tissue from normal weight mice. Protein expression for IGF-IR and ATGL was not increased in obese adipose tissue (FIGS. 2C and 2D).

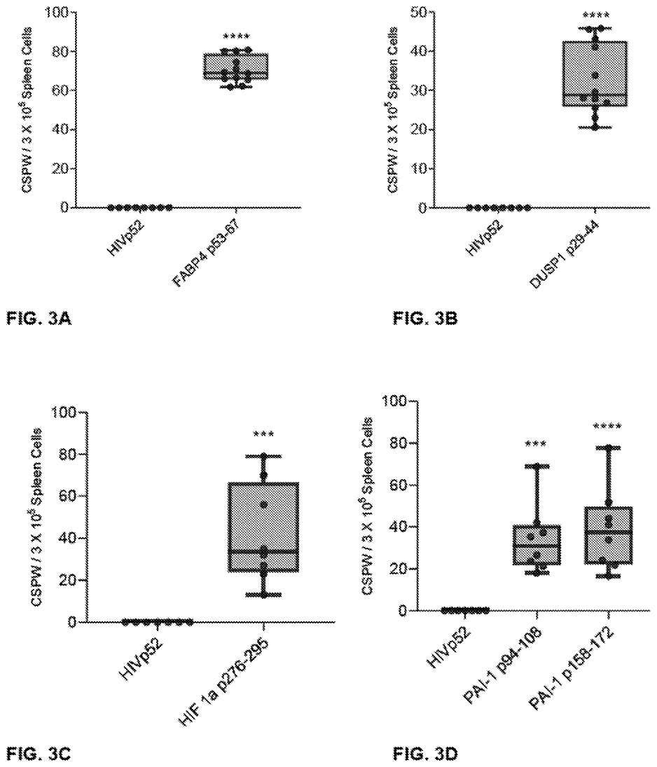

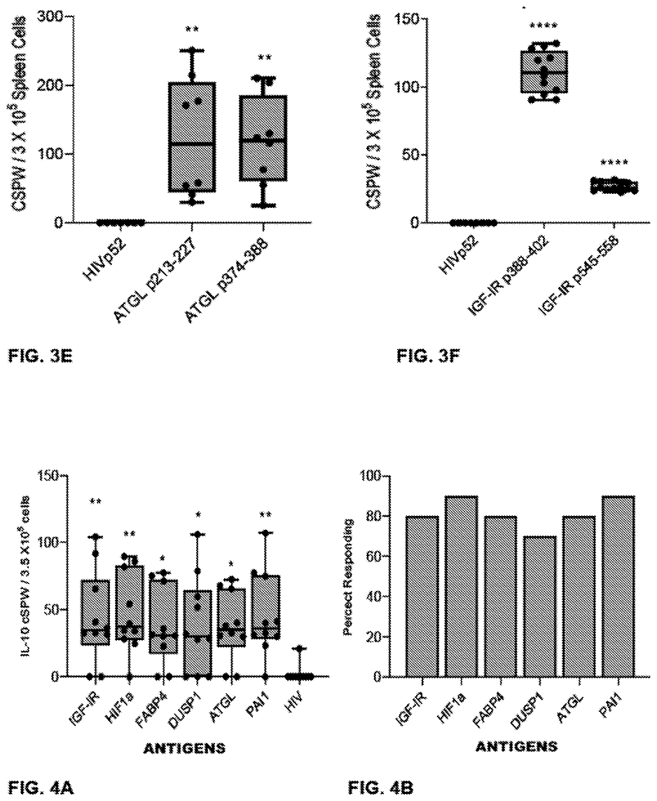

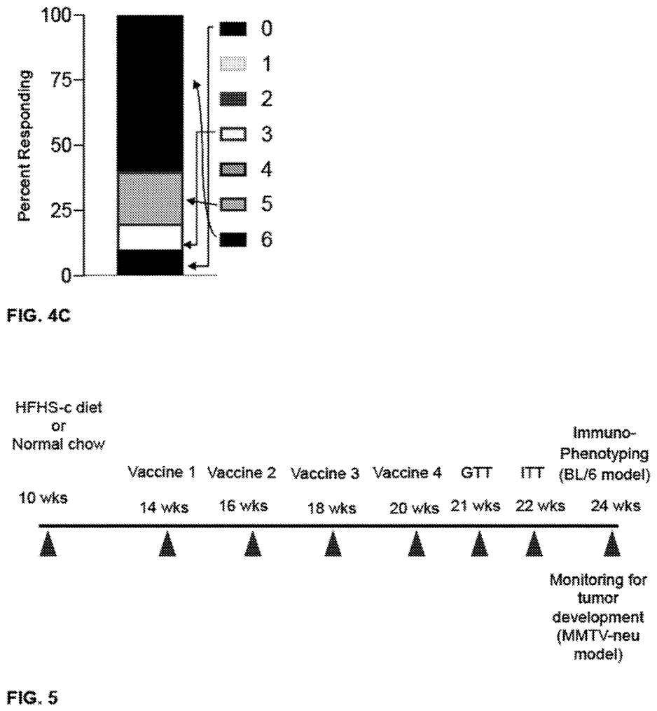

The multi-antigen adipose directed vaccine generated a selective IL-10 response to all antigens in the majority of mice. We initially verified that individual epitopes from each antigen, highly homologous between mouse and human (Table 4), could generate an IL-10 with little to no IFN-γ response after immunization. There were significantly more IL-10-secreting T cells observed when splenocytes from FABP4-p53-67 vaccinated mice (p<0.0001; FIG. 3A), DUSP1-p29-vaccinated mice (p<0.0001; FIG. 3B) or HIF1a-p276-295 vaccinated mice (p=0.0004; FIG. 3C) were stimulated with the vaccinating epitope as compared to the negative control epitope HIVp52-66. Similarly, vaccination with a pool of PAI-1-p94-108 and PAI-1-p158-172 epitopes (p<0.001 for all; FIG. 3D), a pool of ATGL-p213-227 and ATGL-p374-388 epitopes (p<0.01 for all; FIG. 3E) or a pool of IGF-IR-p545 and IGF-IR-p388 epitopes (p<0.0001 for all; FIG. 3F) generated a significantly increased antigen-specific IL-10 response for each epitope as compared to control. No IFN-γ response was detected with any epitope after vaccination.

TABLE 4
Homology for Candidate Antigens Identified as Vaccine Targets

| Candidate Antigen | % Mouse homology |
|---|---|
| HIF1a | 87% |
| FABP4 | 92% |
| IGF1R | 96% |
| PAI1 | 79% |
| DUSP1 | 96% |
| ATGL | 88% |

Immunization with multi-antigen adipose direct vaccine, which we refer to as AdVac, generated a significant IL-10 response for each antigen as compared to the negative control epitope (p<0.01 for all; FIG. 4A). There was no antigenic competition with AdVac. The median incidence of response to any antigen was 80% (range 70-90%; FIG. 4B). The majority of mice responded to all antigens. Sixty percent of mice induced a significant IL-10 response to all six antigens, while an additional 20% responded to a total of five antigens (FIG. 4C).

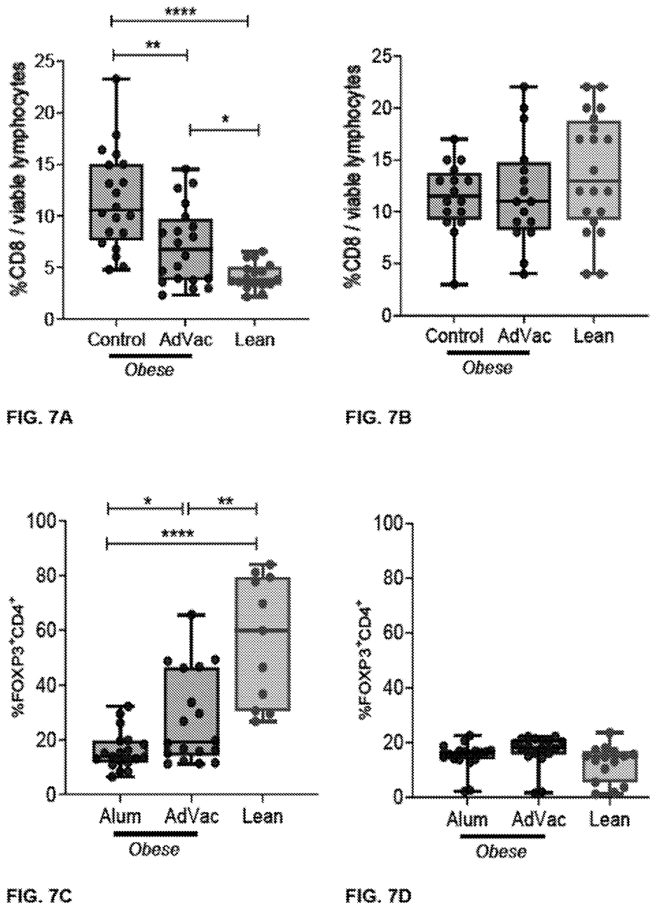

AdVac reduced CD8+ T cell and increased T-regulatory cell levels in the adipose tissue of obese mice. An evaluation of adipose infiltrating T-cell phenotypes in obese and lean individuals revealed Th1 and CD8 T-cells predominate in obese fat and are associated with insulin resistance. Furthermore, IL-10-secreting T-regulatory cells (Treg), which have potent anti-inflammatory effects, were present at very low levels (6). Thus, we evaluated CD8+ and Treg populations infiltrating the adipose tissue in immunized obese mice and compared those levels with normal weight mice. Significantly fewer CD8+ T-cells were observed in the adipose tissue of obese mice immunized with the AdVac (mean; 7±0.8%) as compared to the control (11±1.1%; p=0.0011; FIG. 7A). While there were still significantly more CD8+ T cells in the adipose of AdVac-immunized obese mice as compared to the normal weight control (mean, 4.1±0.3% p=0.043), 40% of the AdVac-immunized mice had CD8+ T-cell levels within the mean and 2SD of that observed in the normal weight mice. In contrast, none of the control immunized obese mice had T-cells within this range. The decrease in CD8+ T-cells after vaccination was specific for adipose tissue as no change in this T-cell population was observed in matched spleen (FIG. 7B). Though immunization with the AdVac did not increase Treg in the adipose tissue of obese mice to the levels detected in normal weight mice (56±5.6%; p=0.0062), there was a significant increase in this T-cell population in the adipose tissue of mice immunized with

AdVac (27±3.7%) as compared to control mice (16±1.5%; p=0.031; FIG. 7C). No changes in Treg levels were observed after vaccination in matched spleen (FIG. 7D).

AdVac increased glucose sensitivity and reduced insulin resistance in obese mice. AdVac did not alter serum cholesterol, triglyceride or IL-6 levels as compared to the control immunized mice. However, AdVac increased glucose sensitivity. Blood glucose concentrations were significantly lower at all time points evaluated in a GTT for the AdVac-immunized obese mice as compared to the control obese mice (p<0.01 for all; FIG. 6A). Additionally, the AUC for AdVac-immunized mice was expectedly lower than the control immunized obese mice (p<0.0001), but was significantly higher than the AUC in the normal weight mice (p<0.0001; FIG. 6B). A similar result was observed for an insulin tolerance test (ITT). Blood glucose was lower at all time points during the study in the mice treated with AdVac (p<0.001 for all; FIG. 6C). The AUC for the mice immunized with AdVac was significantly decreased as compared to the control obese mice (p<0.0001), but was nevertheless higher than measured for the normal weight mouse (p<0.0001; FIG. 6D).

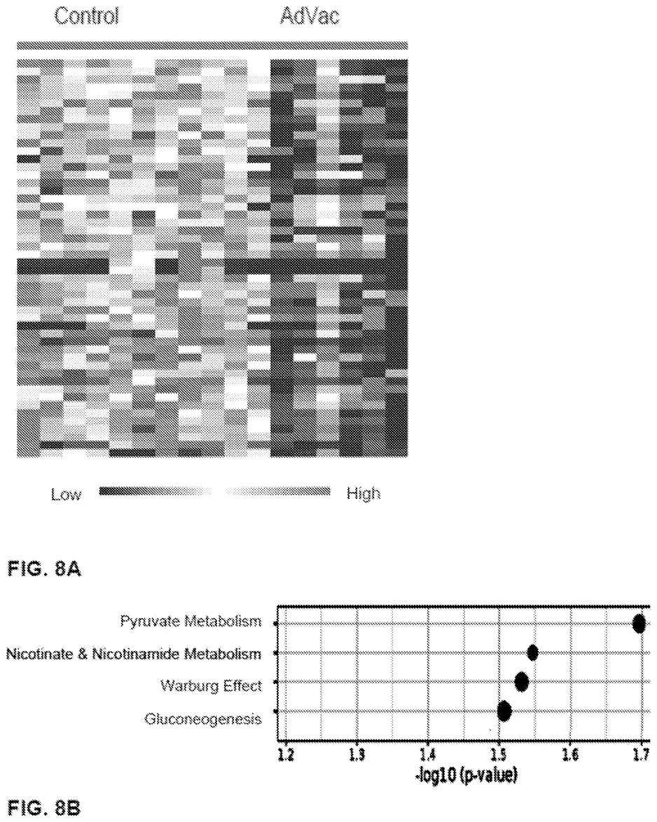

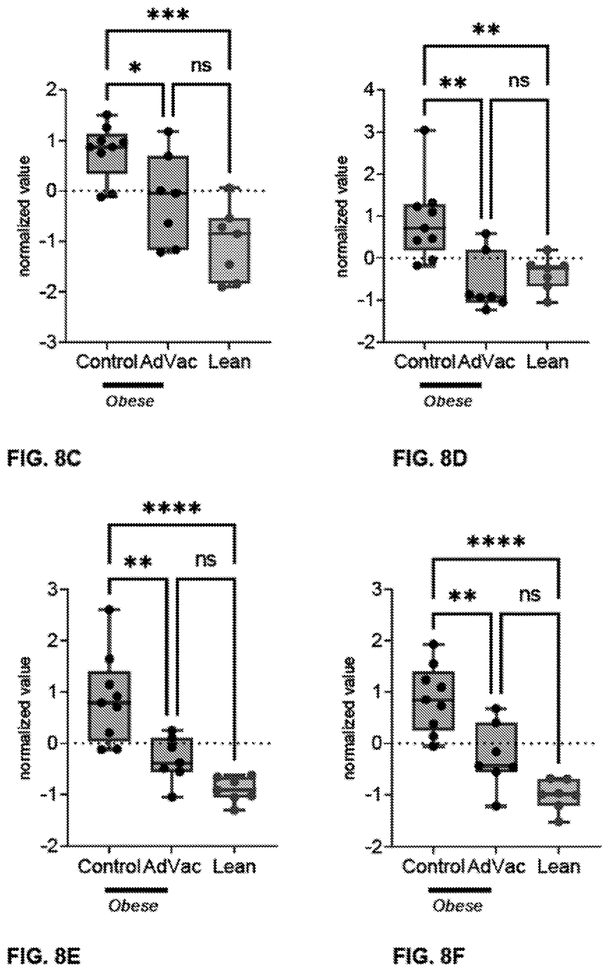

AdVac decreased metabolites in adipose tissue associated with glucose insensitivity and insulin resistance. Adipocytes have been shown to be regulators of glucose homeostasis and can modulate systemic energy balance through alterations in their own metabolism (Rosen, et al. Nature. 2006; 14; 444(7121):847-53). We next questioned if immunization induced changes in the metabolome or lipidome of the adipose tissue. While only two lipids were significantly lower in the adipose of the immunized mice, the levels of 50 aqueous metabolites were significantly lower in tissue from mice immunized with ADVac as compared to control (FIG. 8A). The top four pathways modulated by vaccination as compared to control (p>-log 1.5; FIG. 8B) demonstrated a decrease in metabolites associated with glycolysis, an important pathway in cancer cell metabolism and growth. In the Warburg effect, it has been observed that cancer cells do not produce energy normally through oxidative phosphorylation but rather through aerobic glycolysis, taking up high levels of glucose, followed by lactic acid fermentation. Pyruvate is the terminal product of glycolysis and nicotinamide adenine dinucleotide is an important coenzyme that regulates glycolysis. Gluconeogenesis results in the generation of glucose from non-carbohydrate carbon substrates and may aid in providing the high levels of glucose needed for the abnormal metabolism of cancer cells. An increase in glycolysis has been shown to be a key metabolic pathway promoting metastasis in breast cancer. Of note, after vaccination, there were four metabolites in the top pathways that were no different than the levels observed in lean untreated mice (FIG. 8C-8F).

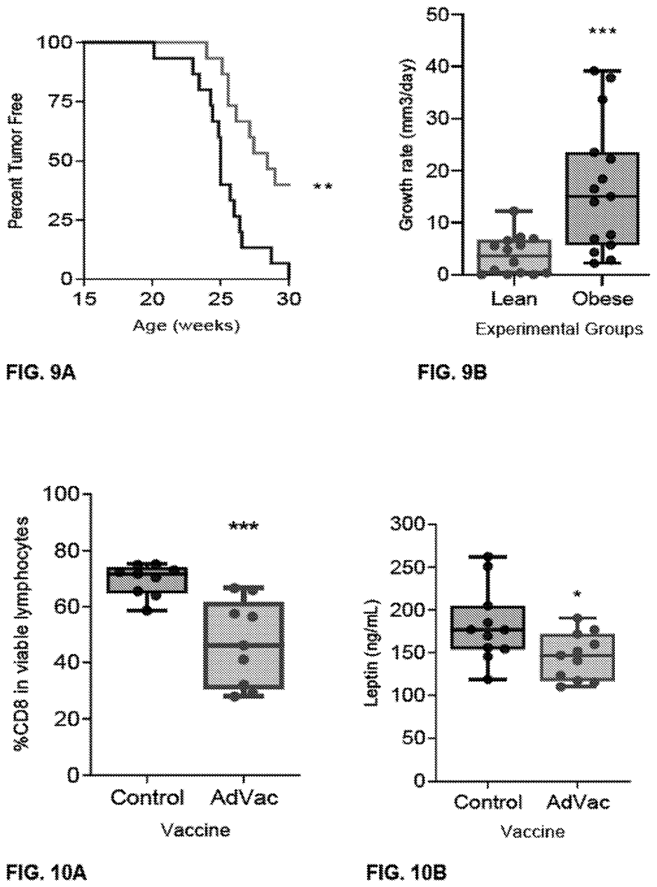

Obese TgMMTV-neu develop a greater number of tumors with a faster growth rate than lean TgMMTV-neu mice. FIG. 9A is a Kaplan-Meier curve demonstrating percent tumor free in obese (lower line) and lean (upper line) mice at 30 weeks. Tumor growth rate (mm3/day) for lean or obese mice is shown in FIG. 9B.

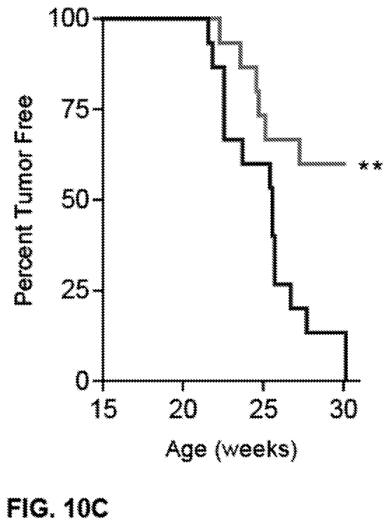

AdVac restores insulin sensitivity, reduces systemic leptin levels and results in the development of fewer tumors in obese Tg-MMTV-neu mice. Similar to the diet induced obesity (DIO) model, we observed increased glucose sensitivity in the AdVac-immunized group as compared to the control in obese Tg-MMTV-neu mice. A GTT revealed that the glucose level in the blood before the test was significantly lower in the immunized group as compared to the control (p<0.0001). The immunized group exhibited less insulin resistance than the control as there was significantly less glucose detected in the blood at every time point measured (p<0.0001 for all). After the obese mice were immunized, significantly fewer CD8+ cells were observed in the mammary adipose tissue as compared to the control obese (p=0.001; FIG. 10A). There was significantly less leptin detected in the serum of mice vaccinated with ADVac as compared to control (Alum; p=0.024; FIG. 10B). The median age of tumor development was 25 weeks in the control cohort and 29 weeks in the ADVac-immunized group (p=0.009; FIG. 10C). Sixty percent (9/15) of the vaccinated mice were tumor free at the planned study termination, whereas 100% of the control mice had developed tumor before the study endpoint.

### REFERENCES

1. Disis M L, et al. J Immunol Methods. 2006; 308:13-8.
2. Park K H, et al. Cancer Res. 2008; 68:8400-9.
3. Cecil D L, et al. Cancer Res. 2014; 74:2710-8.
4. Cecil D L, et al. Clin Cancer Res. 2017; 23:3396-404.
5. Cecil D L, et. al. J Immunother Cancer 2021 March;9 (3): e002355
6. Mclaughlin T, et al. Arterioscler Thromb Vasc Biol. 2014; 34:2637-43.

### Example 3: Confirming the Efficacy and Safety of a Breast Cancer Prevention Vaccine Targeting Inflammatory Adipocytes

Two transgenic mouse models can be used to validate the efficacy of ADVac. First, one can use the TgMMTV-neu mouse, a model of luminal B breast cancer.7,8 Since mice are not born with lesions, the goal is primary breast cancer prevention. The second model is the TgC3(1)-Tag (C3T) representing “basal like” TNBC.⁹ C3T mice are born with DCIS, thus, vaccination would represent breast cancer interception. We have shown feeding TgMMTV-neu mice a HFHS diet leads to insulin resistance and acceleration of mammary cancer onset, with a median age of tumor development of 25 weeks in the HFHS cohort and 28.4 weeks in the normal chow cohort (p=0.0011). HSHS diet fed mice had a faster tumor growth (mean, 12.1 mm³/day) compared to tumors from lean mice (mean, 4.2 mm3/day; p=0.004 and increased tumor multiplicity.

TABLE 5
Experimental Groups

| Group | | |
|---|---|---|
| Number | Treatment | Expected Outcome |
| 1 | TgMMTV-neu mice, normal diet | Lean, 100% develop breast |
| | | cancer |
| 2 | TgMMTV-neu mice, HFHS diet | Obese, 100% develop |
| | | aggressive breast cancer |
| 3 | TgMMTV-neu mice, HFHS diet, | Obese, 100% develop |
| | control vaccine | aggressive breast cancer |
| 4 | TgMMTV-neu mice, HFHS diet, | Obese, 50% protected 50% |
| | ADVac | with breast cancer |
| 5 | Age matched FVB/n mice, | Obese, used as control to |
| | HFHS diet, ADVac | document immunity |

In the TgMMTV-neu model, we will evaluate 5 experimental groups (Table 5). We include Group 5 to document that antigen specific IL-10 secreting T-cells are maintained during active immunization and to assess the immunologic efficacy of vaccine boosters. We will use antigen specific T-cell lines from Group 5 to determine the specific T-cell receptors used to recognize the antigens in ADVac. Defining specific TCRVbeta (b) will allow us to track the distribution of vaccine induced T-cells. Mice receiving ADVac will be immunized with 100 μg of each epitope 4 times at 10-14-day intervals starting when the mice become obese. Booster vaccines will be given every 6 weeks until study end. Mice will be sacrificed when their tumor reaches 1000 mm³ or at 52 weeks if tumor free. We hypothesize that we will be able to duplicate a 60% protection rate. Secondary endpoints include the rate of tumor growth in mice developing tumors to ensure ADVac does not accelerate tumor proliferation and multiplicity of mammary tumor development. Spleens will be collected at the end of the experiment to assess ADVac antigen specific immunity using IL-10 ELISPOT. Samples of visceral and mammary adipose tissue and tumors will be collected and stored. We will include 30 mice per group. Employing a similar experimental design as described for the TgMMTV-neu model, we will test ADVac immunization in the C3T mouse model using a similar schema for the groups. The C3T tumors develop earlier and grow faster than those in the TgMMTV-neu so we will begin vaccination 1 week after starting the animals on the specific diets. The anticipated primary outcome is that we would prevent tumor in 25% of vaccinated mice.¹⁰ Similar analysis will be performed as described for the TgMMTV-neu.

Immune cells play a critical role in the propagation of adipose inflammation and generation of metabolic dysfunction.¹¹ We observe significant differences in the incidence of palpable tumors in the TgMMTV-neu mice immunized with ADVac at 31 weeks. For this reason, we will add 10 mice to TgMMTV-neu groups 1~4 described above to collect visceral and mammary adipose and tumor at 31 weeks for the experiments described below. We expect samples from the following mice cohorts; (1) tumor bearing lean, (2) tumor bearing obese, (3) tumor bearing obese given the adjuvant control vaccine, (4) ADVac immunized obese protected from tumor growth, and (5) ADVac immunized obese developing tumor despite vaccination.

We will use mass cytometry (CyTOF) to evaluate vaccine induced changes in immune cells subsets and activation status. Using broad and unbiased cellular profiling, our goal will be to discern differences in cellular compositions or phenotypes between vaccine protected and unprotected mice. We will also have material from tumor bearing lean and tumor bearing obese mice as comparisons. The Newell lab has extensive experience in the use of mass cytometry to study various tissues and cell types including multiple tumor types in both mouse and human.^{12,13} To greatly reduce batch effects, normalization beads¹⁴ and frozen aliquots of antibody cocktail¹⁵ will be used. We will employ the 10× Genomics single-cell sequencing-based multiomics approach to more deeply compare the protein and gene expression profiles between protected and unprotected mice. For these experiments, cells will be stained with a cocktail of CITE-seq antibodies (Biolegend, inclusive of all markers in the mass cytometry panel) prior to live cell FACS sorting and analysis using the 5′ 10× Genomics immune profiling platform.

We will perform metabolic analysis on visceral and adipose tissue as described to identify significant differences between ADVac protected and unprotected mice. Systems level analysis of cellular metabolic function will be conducted by measuring glucose and fatty acid energy metabolism in real-time using the Agilent Seahorse XF 96 extracellular flux analyzer. Direct ex vivo samples of purified CD4 and CD8 T cells will be isolated from mammary fat, visceral fat, tumors and spleens of obese mice immunized with alum vaccine or ADVac and compared with purified T-cells from matching tissues isolated from unimmunized obese and lean control mice. Oxygen consumption rate (OCR) and extracellular acidification rate (ECAR) will be measured as read-outs of mitochondrial respiration and glycolysis, respectively by the Kalia laboratory. The relative functional relevance of ECAR and OCR in will be confirmed by forcing the cell to respire using glucose analog 2-deoxy glucose (2-DG) or oligomycin (ATP synthase inhibitor) and rotenone+antimycin A (complex I and II inhibitors) to shut down mitochondrial respiration. Trifluoromethoxy carbonylcyanide phenylhydrazone will be used to drive the respiratory chain to function at maximum capacity by collapsing the proton gradient for measuring the cellular spare respiratory capacity (SRC) over basal OCR. Etomoxir will be used to inhibit fatty acid oxidation for further confirmation of fatty acids as the primary nutrient source. Metabolic functions of T-cells will be correlated with the metabolites in corresponding tissues.

### REFERENCES

7. Guy, C. T., et al. Proc Natl Acad Sci USA 89, 10578-10582 (1992).
8. Herschkowitz, J. I., et al. Genome Biol 8, R76 (2007).
9. Maroulakou, I. G., et al. Proc Natl Acad Sci USA 91, 11236-11240 (1994).
10. Gad, E., et al. Breast Cancer Res Treat 148, 501-510 (2014).
11. Turbitt, W. J., et al. Immunol Rev 295, 203-219 (2020).
12. Li, S., et al. Proc Natl Acad Sci USA 118 (2021).
13. Cheng, Y., et al. Immunity 54, 1825-1840 e 1827 (2021).
14. Finck, R., et al. Cytometry A 83, 483-494 (2013).
15. Schulz, A. R., et al. Cytometry A 95, 910-916 (2019).

Throughout this application various publications are referenced. The disclosures of these publications in their entireties are hereby incorporated by reference into this application in order to describe more fully the state of the art to which this invention pertains.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A nucleic acid molecule comprising a nucleic acid sequence encoding a polypeptide, wherein the polypeptide comprises comprising at least two epitopes selected from the group consisting of:
(a) amino acids 53-67 of Fatty acid-binding protein 4 (FABP4);
(b) amino acids 29-43 of Dual Specificity Phosphatase 1 (DUSP1);
(c) amino acids 94-108, 120-134, 158-172, 186-200, 270-284 and/or 288-302 of Plasminogen activator inhibitor-1 (PAI-1);
(d) amino acids 93-107, 213-227 and/or 374-388 of Adipose triglyceride lipase (ATGL);
(e) amino acids 276-295 of Hypoxia-inducible factor 1-alpha (HIF1a);
(f) amino acids 388-402 and/or 545-558 of Insulin like growth factor1 receptor (IGF-1R); and
(g) an amino acid sequence having at least 90% identity with any one of the foregoing.

2. The nucleic acid molecule of claim 1, wherein the polypeptide is selected from amino acids 53-67 of FABP4, amino acids 29-43 of DUSP1, amino acids 276-295 of HIF1a, and a combination thereof.

3. The nucleic acid molecule of claim 1, wherein the polypeptide comprises amino acids 94-108 and/or 158-172 of PAI-1, amino acids 213-227 and/or 374-388 of ATGL, amino acids 388-402 and/or 545-558 of IGF-1R, or a combination thereof.

4. The nucleic acid molecule of claim 1, wherein the polypeptide comprises at least one of each of (a), (b), (c), and (d).

5. The nucleic acid molecule of claim 1, wherein the polypeptide comprises each of:
(a) amino acids 53-67 of FABP4;
(b) amino acids 29-43 of DUSP1;
(c) amino acids 94-108 and 158-172 of PAI-1;
(d) amino acids 213-227 and 374-388 of ATGL;
(e) amino acids 276-295 of HIF1a; and
(f) amino acids 388-402 and 545-558 of IGF-1R.

6. The nucleic acid molecule of claim 1, wherein the polypeptide comprises at least one of each of (a), (b), (c), (d), (e), and (f), and optionally further comprises a linker sequence disposed between the epitopes.

7. The nucleic acid molecule of claim 6, wherein the linker sequence is selected from: GPGPG and GGGS.

8. (canceled)

9. The nucleic acid molecule of claim 1, wherein the nucleic acid sequence comprises a heterologous sequence.

10. The nucleic acid molecule of claim 9, wherein the heterologous sequence encodes a promoter, a transcriptional start site, a translational start site, a detectable marker, a mRNA processing splice site, a polyadenylation sequence, and/or a regulatory element.

11. A vector comprising the nucleic acid molecule of claim 1, wherein the vector is capable of directing expression of the encoded polypeptide(s).

12. The vector of claim 11, wherein the vector is a DNA plasmid, messenger RNA (mRNA), or viral vector.

13. The vector of claim 12, wherein the viral vector is a lentiviral vector, an adenoviral vector, or a poxviral vector.

14. (canceled)

15. A polypeptide comprising at least two epitopes selected from the group consisting of:
(a) amino acids 53-67 of Fatty acid-binding protein 4 (FABP4);
(b) amino acids 29-43 of Dual Specificity Phosphatase 1 (DUSP1);
(c) amino acids 94-108, 120-134, 158-172, 186-200, 270-284 and/or 288-302 of Plasminogen activator inhibitor-1 (PAI-1);
(d) amino acids 93-107, 213-227 and/or 374-388 of Adipose triglyceride lipase (ATGL);
(e) amino acids 276-295 of Hypoxia-inducible factor 1-alpha (HIF1a);
(f) amino acids 388-402 and/or 545-558 of Insulin like growth factor1 receptor (IGF-1R); and
(g) an amino acid sequence having at least 90% identity with any one of the foregoing.

16. A composition comprising the polypeptide of claim 15, wherein the polypeptide comprises at least one of each of (a), (b), (c), (d), (e), and (f), and optionally further comprises one or more linker sequences disposed between the epitopes.

17. A composition comprising the polypeptide of claim 15, wherein the composition further comprises an adjuvant.

18. A method for eliciting a Type 2 immune response directed at inflammation related to obesity, for preventing or treating inflammation in obesity, and/or for preventing or treating disease associated with metabolic obesity in a subject, the method comprising administering to the subject the polypeptide of claim 15, or a nucleic acid molecule encoding the same.

19. The method of claim 18, wherein the disease associated with metabolic obesity is cancer.

20. The method of claim 19, wherein the cancer is breast cancer.

21. The method of claim 18, wherein the disease associated with metabolic obesity is non-alcoholic fatty liver disease (NAFLD) or pre-diabetes.

22. (canceled)

23. The method of claim 18, wherein the subject is human.
